(19) United States
(12) Patent Application Publication
(10) Pub. No.: US 2023/0095948 A1
(43) Pub. Date: Mar. 30, 2023

(54) A DEEP TISSUE ULTRASONIC IMPLANTABLE LUMINESCENCE OXYGEN SENSOR

(71) Applicants: The Regents of the University of California, Oakland, CA (US); Chan Zuckerberg Biohub, Inc., San Francisco, CA (US)

(72) Inventors: Michel M. Maharbiz, El Cerrito, CA (US); Soner Sonmezoglu, Berkeley, CA (US)

(21) Appl. No.: 17/799,891

(22) PCT Filed: Feb. 19, 2021
(86) PCT No.: PCT/US2021/018751

§ 371 Date: Aug. 15, 2022

Related U.S. Application Data
(60) Provisional application No. 62/978,703, filed on Feb. 19, 2020.

(51) Int. Cl. A61B 5/00; H04B 11/00

(52) U.S. Cl. CPC A61B 5/0015; A61B 5/0082; H04B 11/00; A61B 2562/0233; A61B 2560/0214

(57) ABSTRACT

The following relates generally to measuring a patients O₂ level with a mote implanted in the patient's tissue. For example, a mote implanted in a patients tissue may be powered by ultrasound (US) signals generated by an ultrasound interrogator that is external to the patient. Components on the mote may be duty cycled off to advantageously decrease power consumption. A luminescence sensor on the mote may be used to measure the O₂ level, and the luminescence sensor may be optically isolated from the patients tissue by an opaque material such as black silicon.

### TECHNICAL FIELD

The present invention relates to implantable devices for sensing and reporting an O₂ level in a subject using ultrasonic backscatter.

### BACKGROUND

Previously known systems for continuous monitoring of regional tissue oxygenation (RTO) provide therapeutic guidance for critical care patients. This allows for a better understanding of health and disease prognosis. For example, blood oxygenation levels are useful in monitoring compartment syndrome, cancer, organ transplants and so forth. However, current technologies for RTO assessment require tethered, wired connections or batteries, creating problems related to implantation and chronic use due to their large volume. What is needed is a smaller implantable device for sensing O₂ concentrations.

### SUMMARY

Described herein are systems and methods for sensing a patient's O₂ level with a device implanted in the patient's tissue, and reporting the sensed O₂ using ultrasonic backscatter. Further described are systems including one or more implantable devices and an interrogator.

In one aspect, there is provided a mote for measuring an O₂ level of a patient, the mote comprising: a mote piezo configured to both send and receive ultrasound (US) waves; a capacitor configured to be powered by the conversion of US waves received by the mote piezo to electrical energy; and a luminescence sensor configured to be powered by the capacitor, wherein at least part of the luminescence sensor is optically isolated by an opaque material.

In some embodiments of the mote, the opaque material is black silicon.

In some embodiments, the optical isolation is optical isolation between the at least part of the luminescence sensor and tissue of a patient.

In some embodiments, the luminescence sensor is entirely optically isolated from tissue of a patient.

In some embodiments of the mote, the luminescence sensor further comprises: a light emitting diode (LED) configured for optical excitation; a biocompatible film configured for encapsulation of O₂-sensitive luminescent ruthenium (Ru) dyes; and an optical filter.

In some embodiments of the mote: the capacitor is part of a mote integrated circuit (IC); the mote IC comprises a low dropout (LDO), a voltage doubler, and a light emitting diode (LED) driver; and the mote IC is configured to: in first phase: (i) power the capacitor by the conversion of the US waves received by the mote piezo to electrical energy, and (ii) duty cycle off at least one of the LDO, voltage doubler and LED driver; and in a second phase: receive an US data transmission.

In some embodiments, the luminescence sensor is configured to measure an O₂ level of a patient based on the US waves received by the mote piezo.

In some embodiments, the capacitor has a value of less than 100 nF.

In some embodiments, the capacitor has a value of 2.5 nF.

In one aspect, there is provided a method for measuring an O₂ level of a patient, the method comprising: in a power up phase, powering a capacitor by receiving an ultrasound (US) signal; and in a data transmission phase, receiving an US data transmission; wherein, during either the power up phase or the data transmission phase, at least one component of a mote is duty cycled off.

In some embodiments of the above-described method, the at least one component of the mote includes at least one of: a low dropout (LDO); a voltage doubler; and a light emitting diode (LED) driver.

In some embodiments of the above-described method, the at least one component of the mote includes all of: a low dropout (LDO); a voltage doubler; and a light emitting diode (LED) driver.

In some embodiments of the above-described method, the method further comprises: transmitting an electrical current generated from the received US data transmission to a luminescence sensor configured to measure the O₂ level of the patient; modulating the electrical current based on the measured O₂ level; transducing the modulated electrical current into an ultrasonic backscatter that encodes the measured O₂ level; and emitting the ultrasonic backscatter to an interrogator.

In some embodiments of the above-described method, the method further comprises, during the data transmission phase: transmitting an electrical current generated from the received US data transmission to a luminescence sensor configured to measure the O₂ level of the patient; and modulating the electrical current based on the measured O₂ level. In some embodiments of the above-described method, the method further comprises, during a backscatter phase: transducing the modulated electrical current into an ultrasonic backscatter that encodes the measured O₂ level; and emitting the ultrasonic backscatter to an interrogator.

In some embodiments of the above-described method, during a backscatter phase: the at least one component of the mote is duty cycled on; and the capacitor discharges to power the at least one component of the mote.

In some embodiments of the above-described method: the mote comprises a luminescence sensor configured to be powered by the capacitor; and at least part of the luminescence sensor is optically isolated by an opaque material.

In some embodiments of the above-described method: the mote comprises a luminescence sensor configured to be powered by the capacitor; and at least part of the luminescence sensor is optically isolated by black silicon.

In some embodiments of the above-described method: the mote comprises a luminescence sensor configured to be powered by the capacitor; the entire luminescence sensor is optically isolated; and at least part of the optical isolation is provided by black silicon.

In some embodiments of the above-described method, the method further comprises exciting an O₂-sensitive luminescent ruthenium (Ru) dye based on the received US data transmission.

In yet another aspect, there is a device for sending and receiving ultrasound (US) signals to a mote, the device comprising: a piezo configured to send and receive ultrasound (US) waves; an US interrogator configured to control the piezo to send and receive the US waves such that: in a power up phase: a power US transmission is made to the mote; and in a data transmission phase: a data US transmission is made to the mote.

In some embodiments of the device, the US interrogator is configured to control the piezo to send and receive the US waves such that no data US transmission is made during the power up phase.

In some embodiments of the device: the piezo is further configured to receive US backscatter; and the US interrogator is configured to analyze the US backscatter to determine a measured amount of O₂.

In some embodiments of the device, the US interrogator is further configured to charge a capacitor of the mote to a predetermined level by controlling the power US transmission.

In some embodiments of the device, the US interrogator is further configured to bring a voltage level of a low drop out (LDO) of the mote to a predetermined voltage level by controlling the power US transmission.

In some embodiments of the device, the US interrogator is further configured to, by controlling the power US transmission, bring: a voltage level of an analog low drop out (A-LDO) of the mote to a predetermined analog VDD (A-VDD) voltage level; and a voltage level of a digital low drop out (D-LDO) of the mote to a predetermined digital VDD (D-VDD) voltage level.

In some embodiments of the device, a luminescence sensor of the mote is optically isolated from a tissue of a patient.

In some embodiments of the device, the data US transmission is configured to cause a luminescence sensor of the mote to excite an O₂-sensitive luminescent ruthenium (Ru) dye.

In yet another aspect, there is a method for measuring an O₂ level of a patient using pulse-echo ultrasound (US) communication, the method comprising: dividing data into a first data packet and a second data packet, wherein the first data packet includes most significant bits and the second data packet includes least significant bits; in a first data transmission phase, transmitting the first data packet; in a second data transmission phase, transmitting the second data packet; and measuring the O₂ level of the patient according to the transmitted first and second data packets.

In some embodiments of the above-described method, the method further comprises: during a first receive backscatter phase, receiving backscatter of the first data packet; and during a second receive backscatter phase, receiving backscatter from the second data packet.

In some embodiments of the above-described method, the method further comprises, prior to the first data transmission phase: in a power up phase, powering a capacitor by transmitting an US signal.

In some embodiments of the above-described method: a preamble precedes the most significant bits of the first data packet.

In some embodiments of the above-described method: a postamble follows the least significant bits of the second data packet.

In some embodiments of the above-described method: the first data packet and the second data packet are each 15 μs long.

In some embodiments of the above-described method: the most significant bits of the first data packet are five bits; and a one bit preamble precedes the most significant bits of the first data packet.

In some embodiments of the above-described method: the least significant bits of the second data packet are five bits; and a one bit postamble follows the least significant bits of the second data packet.

### BRIEF DESCRIPTION OF THE DRAWINGS

FIG. 1A shows an example schematic of an embodiment of a mote including a mote piezo and mote IC.

FIG. 1B illustrates an example mote on the surface of a patient's finger.

FIG. 1C illustrates an example depiction of silica, polydimethylsiloxane (PDMS), O₂, and Ru-dye.

FIG. 1D illustrates an example of an absorption section, stokes shift, optical filter, and emission spectrum.

FIG. 1E illustrates an example of the principle of phase luminometry.

FIG. 1F illustrates an example normalized absorption and emission spectra of the Ru-dye in the O₂-sensing film along with normalized emission spectrum of the blue μLED and transmission spectrum of the optical filter. FIG. 1G illustrates an example current-voltage-light output characteristics of the blue μLED.

FIG. 1H illustrates an example responsivity spectrum of an integrated photodiode with a 300×300 μm² active area and a reverse bias voltage of 0.6 V.

FIG. 11 illustrates an example photobleaching of Ru-dye in the O₂-sensing film under continuous square-wave illumination with a peak excitation light power of ˜1.53 μW at the operating forward current of 24 μA, resulting in an average optical power density of ˜4.9 μW/mm² at the surface of the film, in air (21% O₂) at room temperature for a period of 60 h.

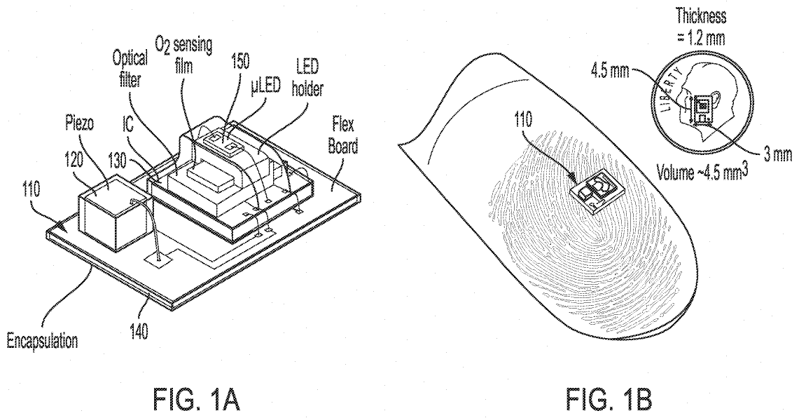

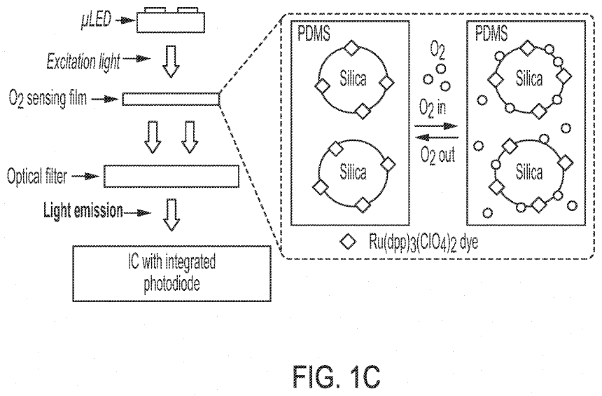

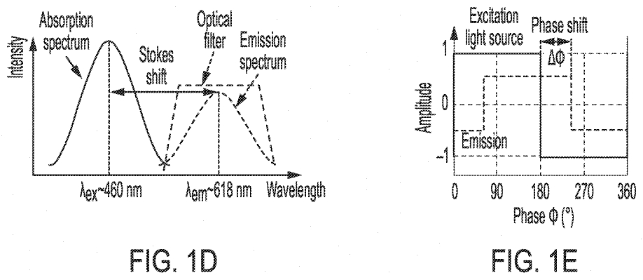

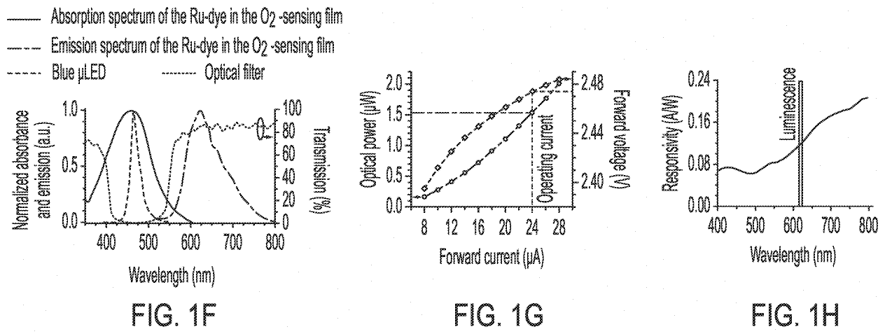

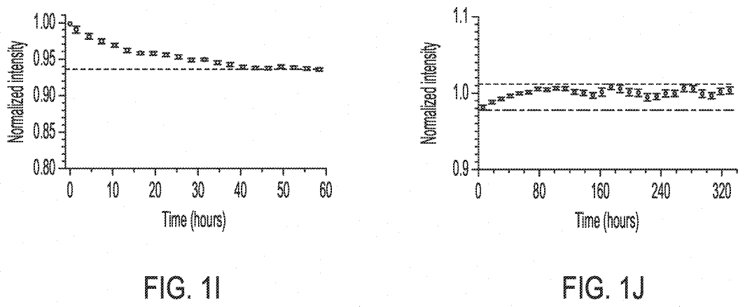

FIG. 1J illustrates an example normalized luminescence intensity of Ru-dye in the PDMS film as a function of time after immersion of the same fully-packaged O₂ sensor used for the photobleaching test in PBS solution at 37° C. in room air.

FIGS. 2A and 2B show an example overall system including an US interrogator in communication with a mote IC.

FIG. 2C shows another example schematic of the IC architecture.

FIG. 3A shows an example timing diagram including various transmission phases. For example, FIG. 3A shows two power up & sensing phases in which an US signal is sent from the US interrogator that is used to generate power for the mote (e.g. used to charge the capacitor Cₛₜₒᵣₑ). In another example, FIG. 3A shows two data transmission phases in which data is transmitted to the mote. In yet another example, FIG. 3A shows a prepare transmission phase. In yet another example, FIG. 3A shows two receive backscatter phases.

FIG. 3B shows another example timing diagram. In particular, FIG. 3B shows an alternative communication protocol that is advantageous for sensors implanted deeper than 5 cm because of the longer ToF to these depths.

FIGS. 4A-4D show an example IC architecture. FIGS. 4A-4D further show detailed views of an exemplary active biasing operational transconductance amplifier (OTA), an exemplary rectifier comparator, and an exemplary LED driver.

FIGS. 5A and 5B show an example of an in vitro setup and wireless measurement of a single O₂ sample, and backscatter relative difference showing ˜14% modulation depth.

FIG. 5C shows an example of backscatter relative difference for 121k O₂ samples.

FIG. 5D shows an example of phase response vs. O₂ concentration.

FIG. 5E shows an example Allan deviation.

FIG. 5F illustrates an example of a measurement recorded with a wireless O₂ sensor operated at 350 samples per second sampling rate and 5 cm depth during the in vitro characterization (e.g., as illustrated in FIG. 5A).

FIG. 5G shows an example backscatter signal from the wireless O₂ sensor captured in an in vivo experiment, showing a high modulation depth of ˜15%.

FIG. 5H depicts an example of a fully implantable, wireless, battery-free luminescence sensor on a balance.

FIG. 51 illustrates an example O₂ sensor response to changes in O₂ concentration in DI water at 37° C. before and after black silicone encapsulation.

FIG. 5J illustrates an example O₂ sensor response to changes in O₂ concentration in DI water at 37° C. before and after ethylene oxide (EtO) sterilization.

FIG. 5K shows data from example O₂ sensors incubated in PBS and undiluted human serum at 37° C. for 10 days.

FIG. 5L illustrates an example of nonlinearity in the phase readout circuitry.

FIG. 5M illustrates an example of a rectifier voltage (V_{rect}) and a voltage doubler output (V_{DC-DC}) during a power-up period of ˜150 μs. At steady-state, the voltage conversion ratio (VCR) of the voltage doubler was 1.91

FIG. 5N illustrates data from an example animal.

FIG. 5O illustrates data from another example animal.

FIG. 5P illustrates an example lifetime (τ)-based Stern-Volmer plot obtained from the data shown FIG. 5F.

FIG. 5Q illustrates an example calibration curve of a wireless O₂ sensor. Calibration of the O₂ sensor in distilled water at 37° C. was conducted by periodically increasing the O₂ concentration of water surrounding the sensor, as shown in FIG. 5F. The dissolved O₂ concentration was measured at each step by a commercial O₂ sensor. The calibration curve was fitted by an exponential equation: pO₂ (mmHg)=A·e^{(B/ϕ)}+C, where ϕ is the sensor phase output, and A, B and C are constant coefficients obtained from the curve fitting. The exponential equation provided high accuracy with an R² of 0.9993. Alternate equations, such as high-order polynomial functions, may be used for calibration curve fitting.

FIG. 5R shows an example of measured impedance of the 10 μm-thick parylene-coated piezo crystal as a function of frequency in distilled water. During system operation, the piezo was driven at 2 MHz frequency, which is close to its open-circuit resonance frequency of 2.05 MHz. The impedance values at 2 MHz provide a good impedance matching with the rectifier input resistance (Rᵢₙ) of ˜11.8 kΩ at the desired (2 V) output voltage of the rectifier, yielding an impedance matching efficiency of ˜97% between the piezo and the rectifier. A capacitive matching network may be used to improve matching efficiency further.

FIG. 5S shows an example normalized acoustic reflection coefficient (Γ) of the piezo crystal versus load resistance (R_{load}), measured with ultrasound at 2 MHz. Here R_{load} simulates Rᵢₙ.

FIGS. 5T and 5U illustrate an example characterization of the external ultrasound transducers, used for measurements at moderate depths ≤5 cm in distilled water using a hydrophone. In particular, FIG. 5T illustrates longitudinal beam patterns; and FIG. 5U illustrates transverse beam patterns.

FIGS. 5V and 5W illustrate example characterization of the external ultrasound transducer, used for measurements at 10 cm depth in distilled water using a hydrophone. Specifically, FIG. 5V illustrates a longitudinal beam pattern; and FIG. 5W illustrates a transverse beam pattern.

FIGS. 6A and 6B show an example system response measured at various dissolved oxygen (DO) concentrations.

FIG. 6C shows an example Allan deviation of example data.

FIG. 6D shows an example response to alternating streams of O₂ and N₂.

FIG. 6E shows an example nonlinearity of a phase readout circuit.

FIG. 6F shows an example Stern-Volmer plot.

FIGS. 7A-7C illustrate an example showing the advantages of the systems and methods disclosed herein over previously known systems.

FIG. 8A illustrates an example system setup without a transverse misalignment.

FIG. 8B illustrates an example of sensor waveform and backscatter signal corresponding to the example setup of FIG. 8A.

FIG. 8C illustrates an example backscatter relative difference corresponding to the setup of FIG. 8A.

FIG. 8D illustrates an example system setup with a transverse misalignment.

FIG. 8E illustrates an example of sensor waveform and backscatter signal corresponding to the example setup of FIG. 8D.

FIG. 8F illustrates an example backscatter relative difference corresponding to the setup of FIG. 8D.

FIG. 8G illustrates an example of sensor placement.

FIG. 8H illustrates another example of waveform and backscatter signal.

FIG. 8I illustrates another example of backscatter relative difference.

FIGS. 9A and 9B show an example system response to various O₂ concentrations. An additional, identical wireless O₂ sensor was also characterized in distilled water at a depth of 5 cm and a sampling rate of 350 samples per second at various O₂ concentrations. This sensor was also used for tissue O₂ monitoring. The data, shown in FIG. 5O, was collected using this sensor. More specifically, FIG. 9A shows phase response vs. time; and FIG. 9B shows phase response vs. dissolved O₂ concentration.

FIGS. 9C, 9D, 9E, and 9F illustrate and example effect of US link alignment on the system operation. FIG. 9C illustrates an example schematic diagram of the misalignment parameters. The measurements were performed in distilled water using a spherically-focused external transducer with a 25.4 mm diameter and a focal depth of 47.8 mm. In this example, the wireless sensor was operated at a fixed ISPTA of 220 mW/cm². FIG. 9D illustrates that the sensor operating while its depth was longitudinally scanned along the central axis of the acoustic field, showing a wide operating window of 16 mm. FIG. 9E illustrates an example where the wireless sensor was placed aligned to the center of the focal plane at the focal depth, and its position and orientation were scanned along transverse and angular directions relative to the central axis of the sensor piezo. FIG. 9F illustrates a map depicted a region where the sensor operates.

FIG. 9G and 9H illustrate example wireless measurements of a single O₂ sample for the sensor operated at 5 cm depth in water with different acoustic intensities. The wireless O₂ sensor was operated with a 2 MHz acoustic wave with an ISPTA of 155 mW/cm² for FIG. 9G, and 478 mW/cm² for FIG. 9H. The minimum rectifier output voltage (V_{rect}) required to operate the sensor was ˜1.36 V for FIG. 9G. The maximum V_{rect} that can be generated by the IC was ˜3 V limited by voltage limiting clamps at the rectifier input to prevent breakdown of the transistors for FIG. 9H.

FIG. 9I shows an example wireless O₂ sensor was at 5 cm depth through a fresh, ex vivo porcine tissue specimen, in which ultrasound waves with 660 mW/cm² de-rated ISPTA, producing an acoustic power of ˜27.67 mW at the external transducer surface, propagated through approximately 2 mm ultrasound gel, 1.5 mm skin, 1 mm fat, and 45.5 mm muscle tissue.

FIG. 9J shows an example sensor waveform and backscatter signal that was captured in the wireless measurement of a single O₂ sample.

FIG. 9K shows an example backscatter relative difference for 118k O₂ samples, showing ˜32% modulation depth. The system achieved a bit error rate (BER) of <10⁻⁵ and a wireless link power transfer efficiency of ˜0.73%. The modulation depth measured ex vivo was lower than the modulation depth measured in vitro in distilled (DI) water (see FIG. 5C) due to a decrease in the ratio of the modulation amplitude (that is, the amplitude difference between the modulated and unmodulated backscatter signals) to the amplitude of the unmodulated backscatter signal. This ratio decrease can be attributed to the ultrasound reflections from internal tissue interfaces that interfered with the total US reflections from the sensor's piezo and the part of the sensor surface at the face of the external transducer. Note that to make a fair comparison, the alignment between central axes of the piezo and the acoustic field was well-tuned by monitoring the rectifier voltage (V_{rect}) amplitude in this measurement and the measurement in DI water.

### DETAILED DESCRIPTION

The present embodiments relate to, inter alia, systems and methods for measuring a patient's O₂ level with a device implanted in the patient's tissue. In particular, continuous monitoring of regional tissue oxygenation (RTO) can provide therapeutic guidance for critical care patients. However, current technologies for RTO assessment require tethered, wired connections or batteries, creating problems related to implantation and chronic use due to their large volume.

In this regard, ultrasound (US) has been demonstrated as an efficient way to wirelessly power and communicate with implantable devices deep in tissue, enabling their miniaturization [see T. C. Chang, et al., “A 30.5 mm³ fully packaged implantable device with duplex ultrasonic data and power links achieving 95 kb/s with <10⁻⁴ BER at 8.5 cm depth,” IEEE ISSCC, 2017, pp. 460-461; see also M. M. Ghanbari, et al., “A 0.8 mm³ ultrasonic implantable wireless neural recording system with linear am backscattering,” IEEE ISSCC, 2019, pp. 284-286] by eliminating the need for wires or large batteries. The systems and methods disclosed herein present a fully wireless implantable, real-time DO monitoring system that combines a luminescence sensor with US technology. Further presented is the first fully wireless implantable luminescence sensor system for deep tissue O₂ monitoring, achieving competitive or better O₂ resolution, the lowest power consumption and the smallest volume (4.5 mm³) of any system previously demonstrated.

By way of overview of some of the electrical power aspects, as somewhat discussed in paragraphs 0069-0070 of WO 2018/009905, which is incorporated by reference herein, and as further somewhat discussed in column 8, line 58 to column 9, line 29, of U.S. Pat. No. 10,300,310, which is also incorporated by reference herein, an implantable device (such as a mote) includes a miniaturized ultrasonic transducer (such as a miniaturized piezoelectric transducer) and a physiological sensor (such as a luminescence sensor). The miniaturized ultrasonic transducer receives ultrasonic energy from an interrogator (which may be external or implanted), which powers the implantable device. The interrogator includes a transmitter and a receiver (which may be integrated into a combined transceiver), and the transmitter and the receiver may be on the same component or different components. The physiological sensor detects a physiological condition (such as pressure, temperature, strain, pressure, or an amount of one or more analytes), and generates an analog or digital electrical signal. Mechanical energy from the ultrasonic waves transmitted by the interrogator vibrates the miniaturized ultrasonic transducer on the implantable device, which generates an electrical current. The current flowing through the miniaturized ultrasonic transducer is modulated by the electrical circuitry in the implantable device based on the detected physiological condition. The miniaturized ultrasonic transducer emits an ultrasonic backscatter communicating information indicative of the sensed physiological condition, which is detected by the receiver components of the interrogator.

A significant advantage of the implantable device is the ability to detect one or more physiological conditions in deep tissue while being wirelessly powered, and to have those physiological conditions wirelessly transmitted to an interrogator, which can be external or relay the information to an external component. Thus, the implantable devices can remain in a subject for an extended period of time without needing to charge a battery or retrieve information stored on the device. These advantages, in turn, allow the device to be smaller and less expensive to manufacture. In another advantage, use of ultrasound allows for the relative time for data communication to be related to distance, which can aid in determining location or movement of the implantable device in real time. Further problems with current technologies include O₂ consumption, susceptibility to biofouling, long readout time, and inability to operate in deep tissue. The systems and methods described herein avoid these problems and others.

More specifically, with reference to FIGS. 1A and 1B, the mote 110 is designed to operate using a single US link for both power and downlink/uplink data transmission; uplink data transmission is performed by digital amplitude modulation of the US backscatter. The use of a single link custom protocol, combined with efficient system-in-package integration and minimal off-chip components, resulted in a very small total mote size (e.g., 4.5 mm³), of great potential for chronic use with minimal tissue damage. The mote 110 is verified to operate safely at 50 mm depth with a resolution <0.76% (5.8 mmHg) across the physiologically relevant O₂ range of 0-13.2% (0-100 mmHg), suitable for in vivo applications, while consuming an average power of 140 μW, including power conversion efficiency. The described system may operate in vitro in distilled water, phosphate-buffered saline (PBS) and undiluted human serum; ex vivo through porcine tissue; and in vivo in an anesthetized sheep model. The ability to monitor tissue oxygenation during physiological states in vivo may be confirmed via surgical implantation deep under the biceps femoris muscle.

The mote 110 may also operate at different depths. In some examples, the mote 110 may operate at centimeter scale depths on an anesthetized sheep (e.g., a large animal). In other examples, the mote 110 operates at greater depths ≥5 cm) through ex vivo porcine (anatomically heterogeneous) tissue.

The mote 110, in the example of FIG. 1A, includes a 750×750×750 μm³ piezo (Lead Zirconate Titanate, PZT) (e.g., a miniaturized piezoelectric transducer that is an ultrasonic transducer) and a luminescence sensor. Regarding the piezo, as mentioned in U.S. Pat. No. 10,300,309, which is incorporated by reference herein, a “piezoelectric transducer” or “piezo” is a type of ultrasonic transceiver comprising piezoelectric material. The piezoelectric material may be a crystal, a ceramic, a polymer, or any other natural or synthetic piezoelectric material.

The luminescence sensor includes a μLED 150 for optical excitation, a biocompatible film for encapsulation of O₂-sensitive luminescent ruthenium (Ru) dyes, an optical filter, and an IC fabricated in a 65 nm LP-CMOS process. In this regard, FIG. 1C illustrates an example depiction of silica, PDMS, O₂, and Ru-dye. In one example implementation, the film thickness (˜100 μm) and the amount of silica particles in PDMS (˜8.3%) were adjusted to maintain a reasonable tradeoff between luminescence intensity, emitted from Ru-dyes under blue-light excitation, and O₂ response time. An example of an O₂-sensitive luminescent ruthenium (Ru) dye is Ru(dpp)₃(ClO₄)₂. The Ru(dpp)₃(ClO₄)₂ complex is advantageous because of its large Stokes shift, relatively long excited state lifetimes, and high photostability. The luminescence sensor achieves a lower power consumption and better O₂ resolution than other sensors at least in part due to the compact integration of sensor components on the IC.

Advantageously, the tissue is optically isolated from the luminescence sensor. To accomplish this, in some embodiments, a particular area of the encapsulation 140 (of FIG. 1A) is made of black silicon or other opaque material to optically isolate the μLED 150 from the tissue. In some embodiments, all components on the mote 110 excluding the piezo 120 are optically isolated using black silicon or other opaque material. In some embodiments, the entire encapsulation 140 is made of black silicon or other opaque material to optically isolate the μLED 150 from the tissue. In some embodiments, only the sensor or part of the sensor is coated in black silicon or other opaque material. In some embodiments, the black silicon advantageously allows the device to avoid background interferences by the luminescence of tissue or blood. In some embodiments, an example fully-packaged sensor (e.g., FIG. 1B) measures 3 mm×4.5 mm×1.2 mm, occupied 4.5±0.5 mm³ volume, and had a detection volume of ˜0.26 mm³ (estimated from the material volume where O₂ molecules diffuse through to the O₂-sensing film under the μLED). The resonant frequency of the piezo determined the carrier frequency of the ultrasound link; as this frequency was set by the crystal thickness and aspect ratio, crystal geometry was chosen to maintain a reasonable tradeoff between the frequency-dependent acoustic loss in tissue, the capacity of power harvesting, and impact on total implant size.

The sensor operates on the principle of phase luminometry, wherein the phase shift (Δϕ) between the excitation and emission signals is monitored to detect O₂ concentration. In one example implementation, upon light excitation at 460 nm, the Ru-dyes emit light at 618 nm, enabling background/excitation light rejection through an optical filter (see, e.g., the example of FIG. 1D). In another example implementation, upon light excitation at 465 nm, the Ru-dyes emit light at 621 nm, enabling background/excitation light rejection through an optical filter. During operation, the Ru-dyes are excited with square wave modulated light at a fixed frequency (fₒₚ), producing an emission at the same fₒₚ, but shifted in phase (ϕ); an example related to this is shown in FIG. E. The phase, ϕ, equals tan⁻¹(2πf_{opτ})≈ω_{opτ} for Ω_{opτ}«1 and is a function of the luminescence lifetime (τ) which in turn is related to local O₂ concentration as τ₀/τ=1+K_{SV}[O₂ ], where τ₀≈6.4 μs is the lifetime at zero O₂ and KSV is the Stern-Volmer constant [see L. Yao, et al., “Sensitivity-enhanced CMOS phase luminometry system using xerogel-based sensors,” IEEE TBioCAS, vol. 3, no. 5, pp. 304-311, October 2009.]. Either intensity or lifetime can be measured to compute dissolved O₂; however, luminescent lifetime (τ) is independent of variations in light source intensity and dye concentration, inner filter effects, and photobleaching (to a wide extent), all of which are main limitations of intensity-based sensors.

In one example implementation, during operation, the emitted light was square-wave modulated at a fixed operating frequency (fₒₚ=20 kHz), exciting the Ru-dyes in the PDMS film with a peak excitation power of ˜1.53 μW, resulting in an average power intensity of ˜4.9 μW/mm² at the film surface (FIGS. 1E and 1G). The excited Ru-dyes produce emission with a typical average power density of ˜8 nW/mm² at 37° C., ˜160 mmHg (room air) O₂ concentration and the same fₒₚ, but with a phase shift (A1) relative to the phase of the excitation light (FIG. 1E). The emission was detected by a 0.6 V reverse-biased, on-chip nwell/psub photodiode with an active area of 300×300 μm² and a responsivity of ˜0.12 A/W at the peak emission wavelength of ˜621 nm after filtering the excitation light using a long-pass optical filter (FIGS. 1C and 1H). The resulting phase shift (Mϕ) equals tan⁻¹(2tfₒpt)≈wₒₚt for wₒₚt«1 and is directly dependent on the luminescence t that, in turn, is related to local O₂ concentration via the Stern-Volmer equation.

Further in this example implementation, photobleaching of the Ru-dye was evaluated using a fully-packaged O₂ sensor (FIG. 1b) continuously operated in room air (21% O₂) at room temperature for a total period of 60 h. The luminescence intensity decreased rapidly to ˜96.8% of its initial value in the first 10 hours and at a slower rate from ˜96.8% to ˜93.6% in the next 50 hours, indicating that the sensor could be operated with a duty cycle of 1%, corresponding to 14.4 min continuous operation in a single day, for 250 days with an only ˜6.4% drop in the luminescence intensity. After the bleaching test, a long-term continuous test (14 days) of the same sensor immersed in phosphate-buffered saline (PBS, 1×) solution at 37° C. was conducted in room air to evaluate dye leaching from the sensing film; the fluctuation in the luminesce intensity was within ±1.7% and did not exhibit a decreasing trend in 14 days.

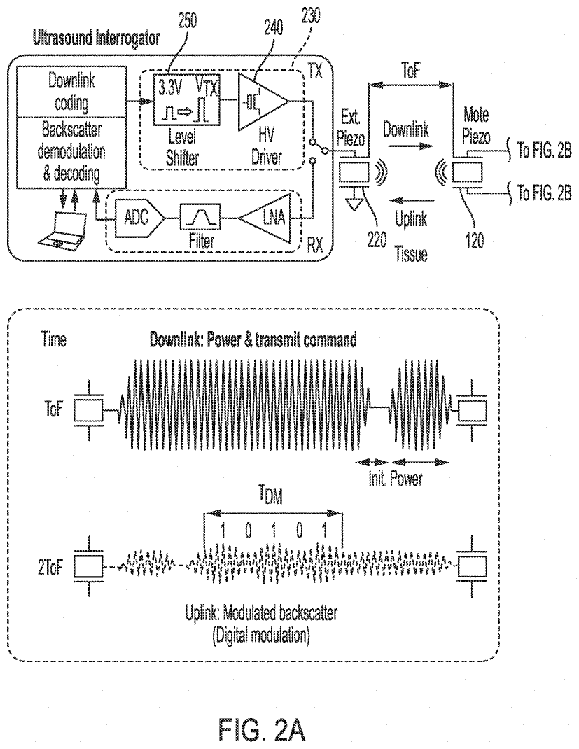

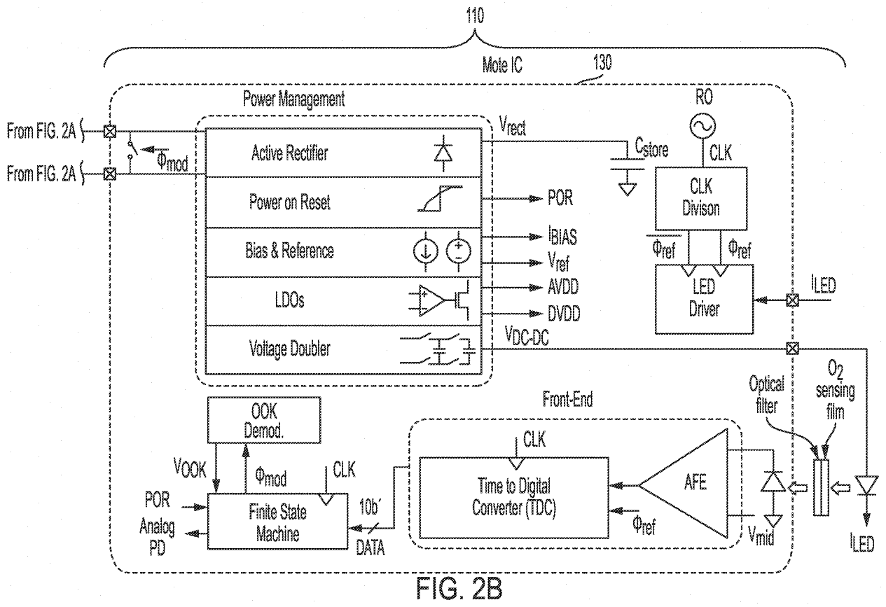

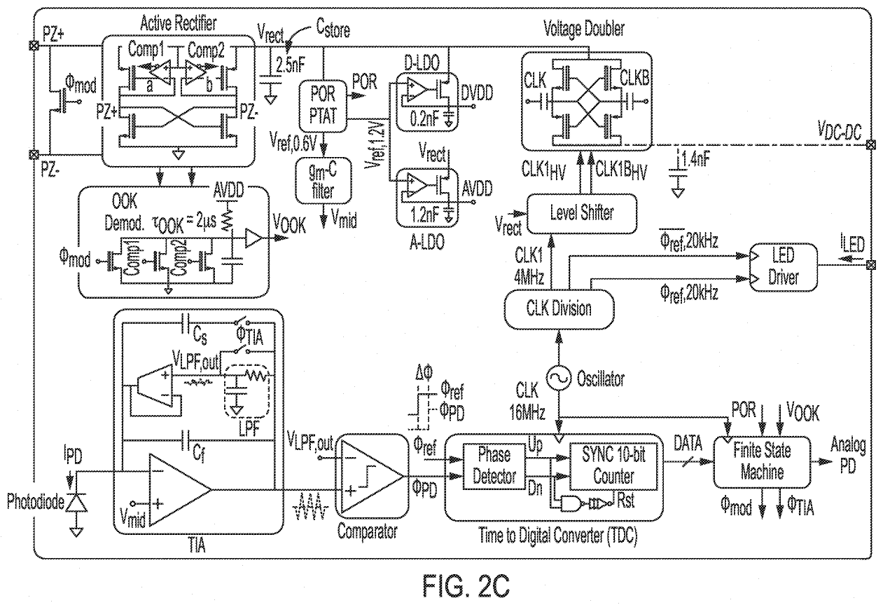

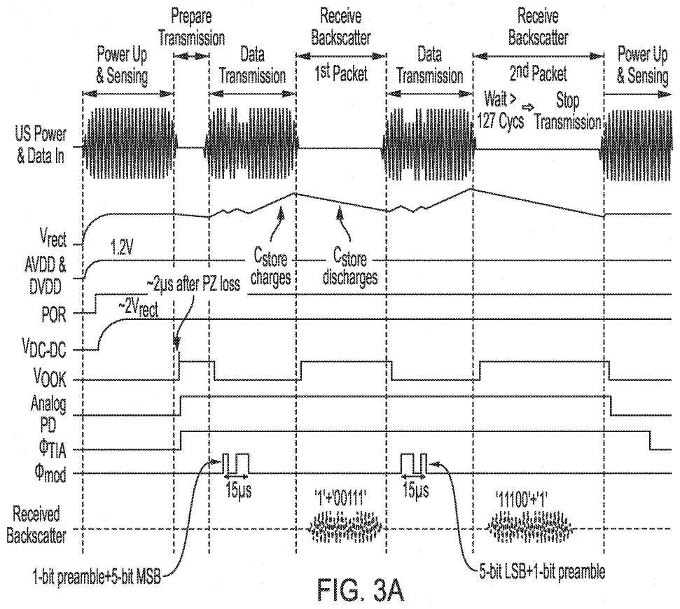

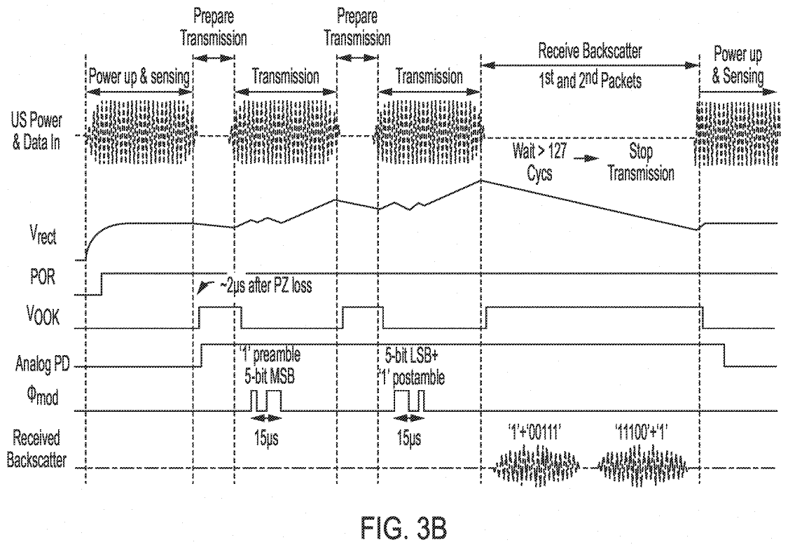

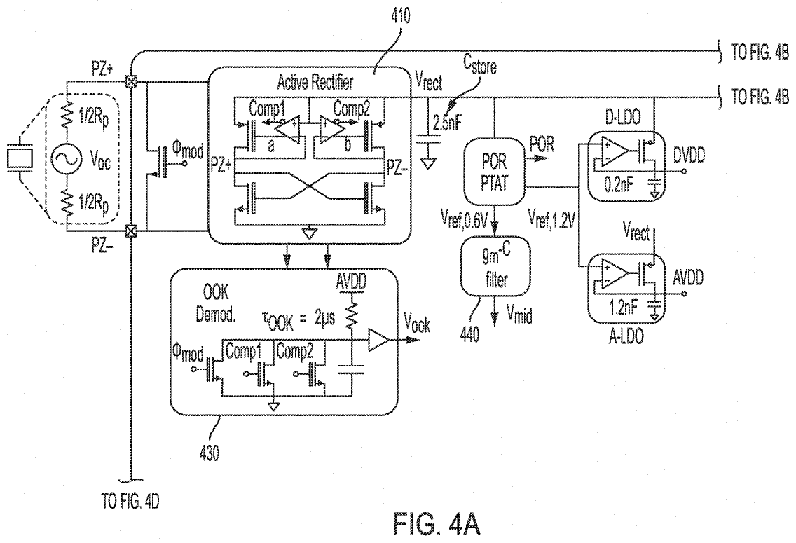

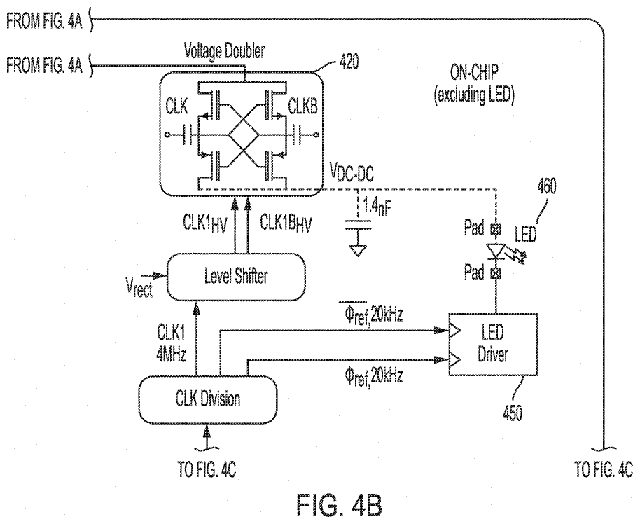

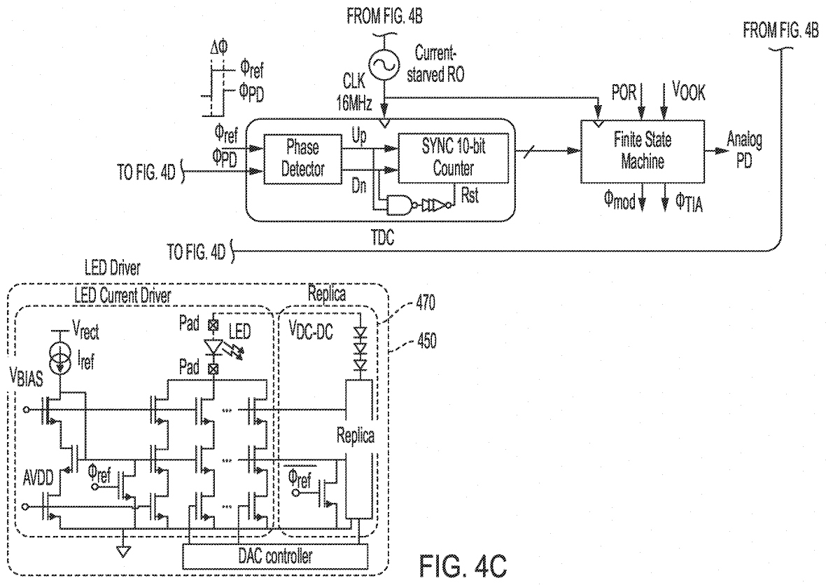

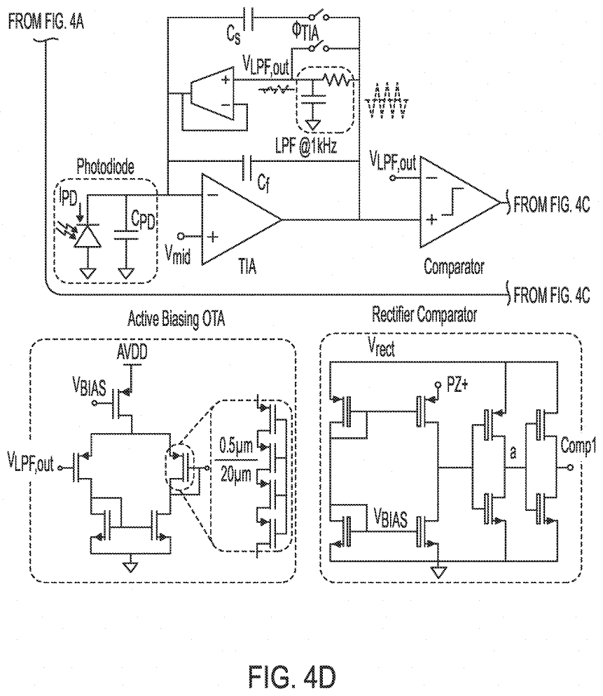

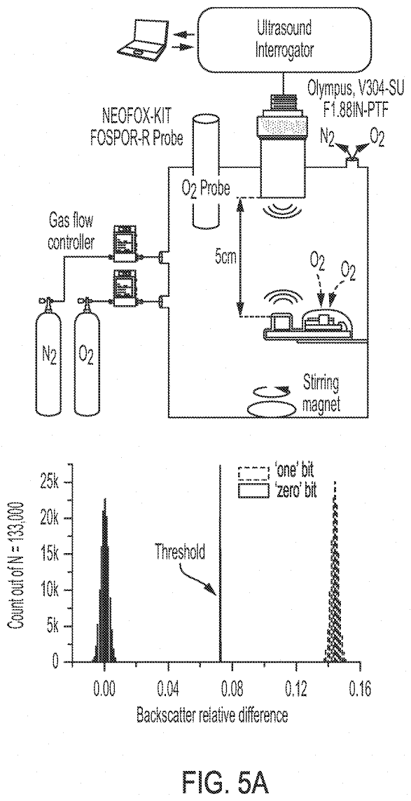

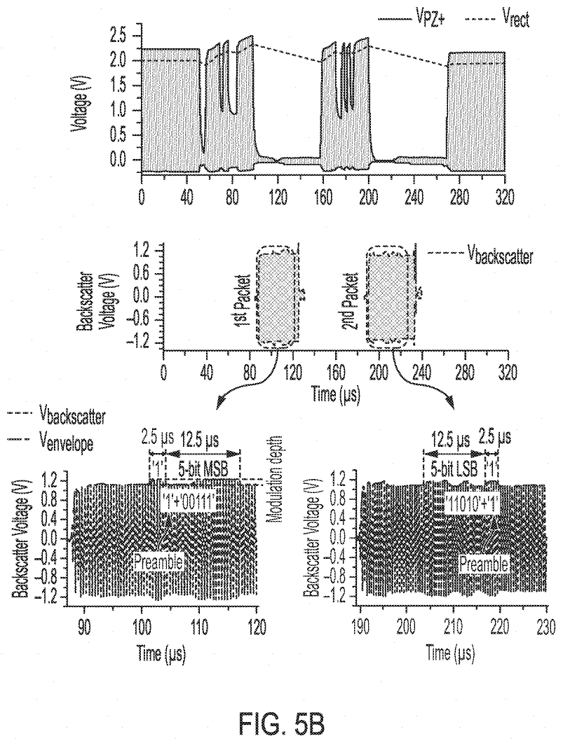

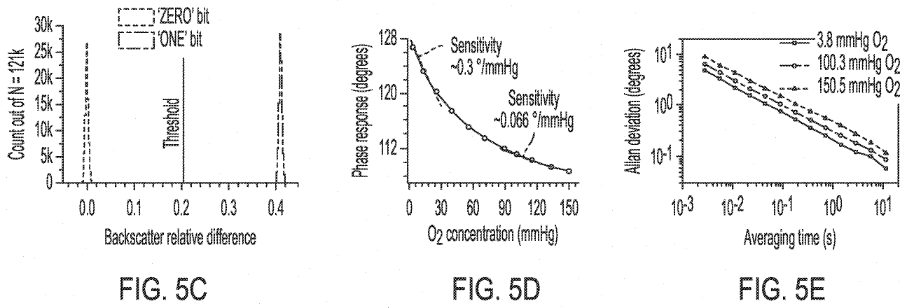

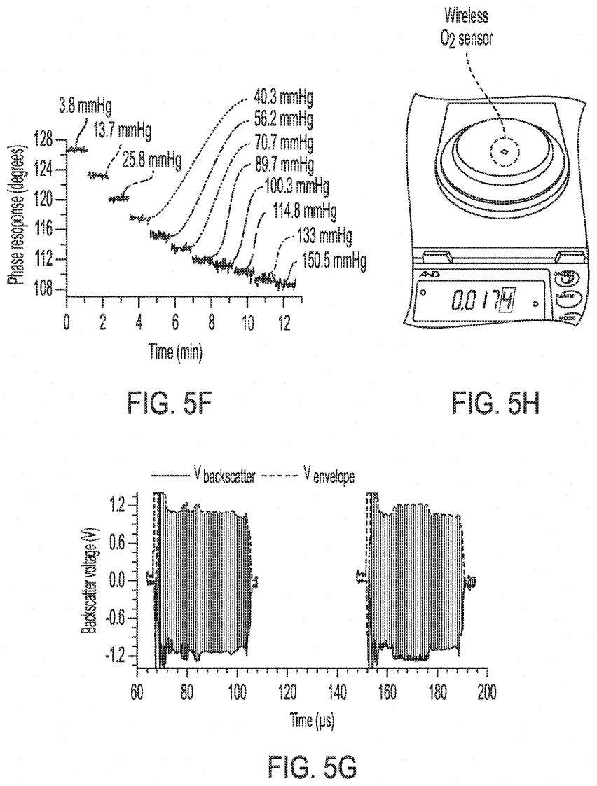

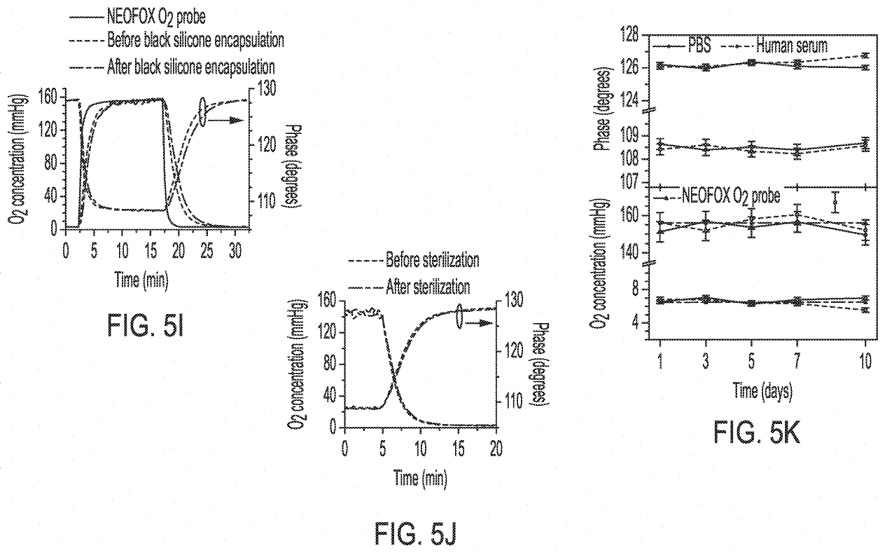

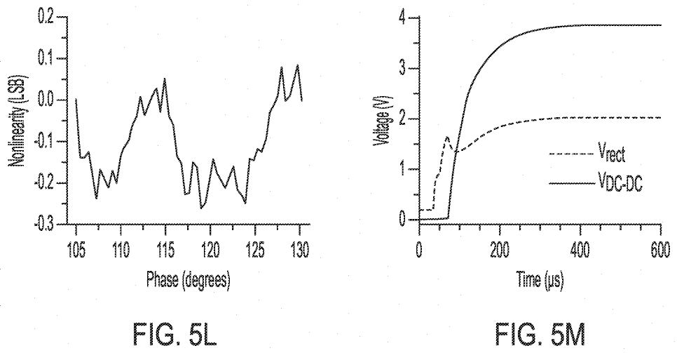

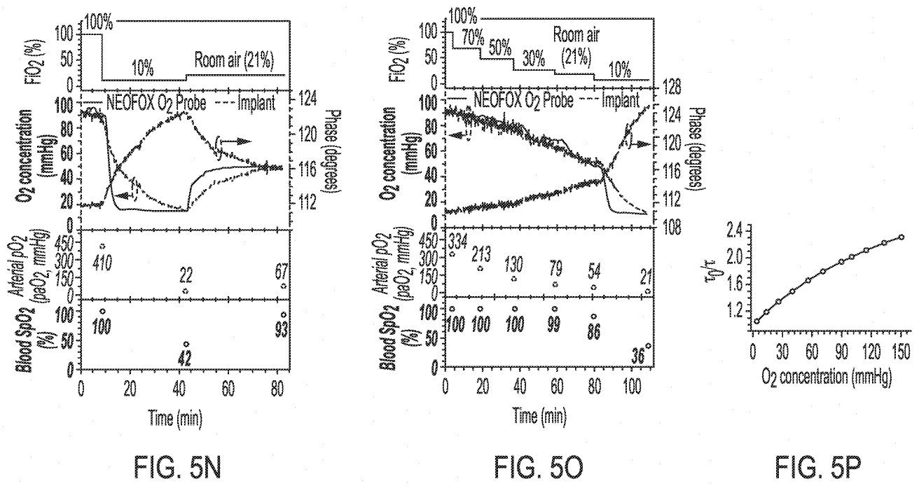

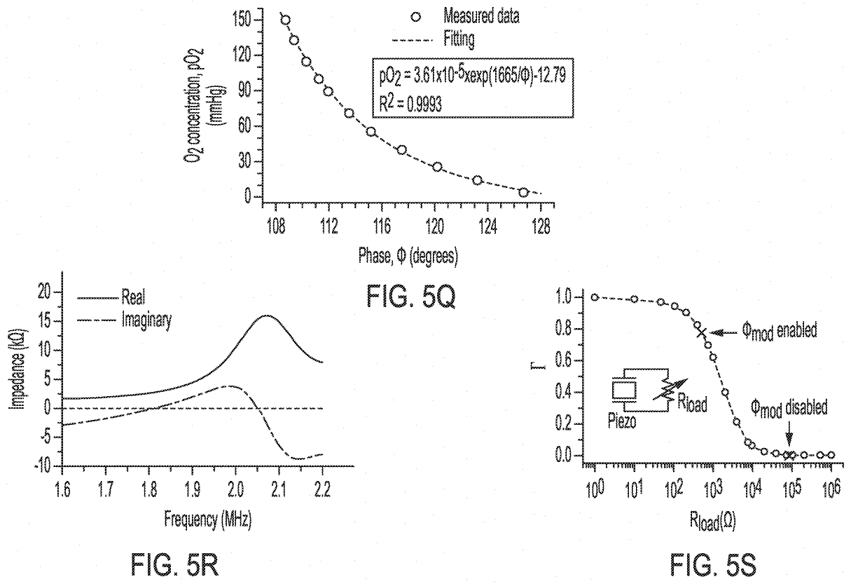

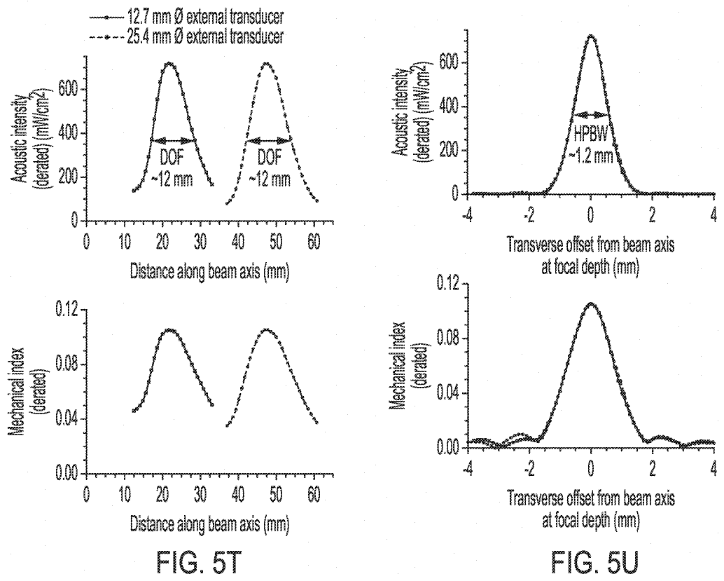

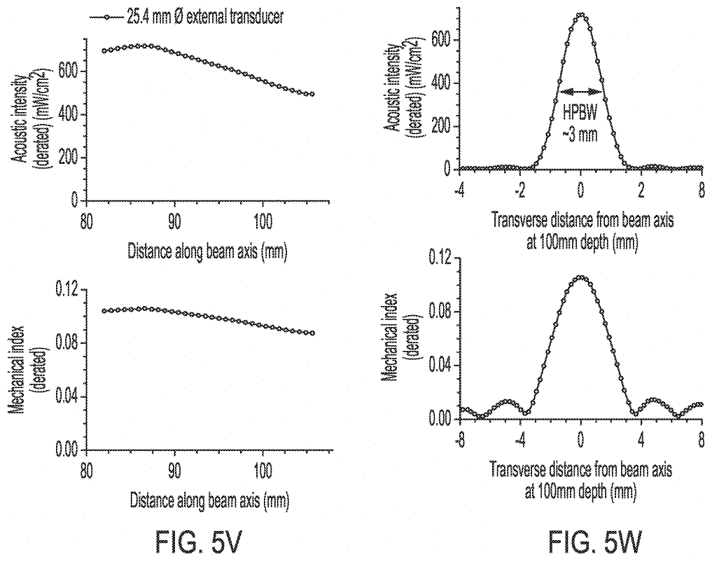

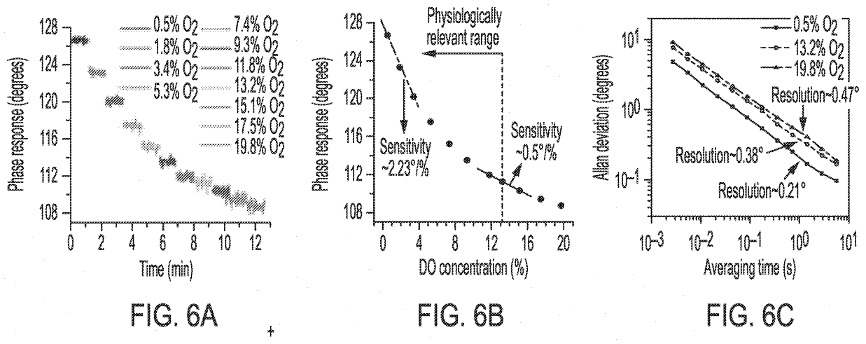

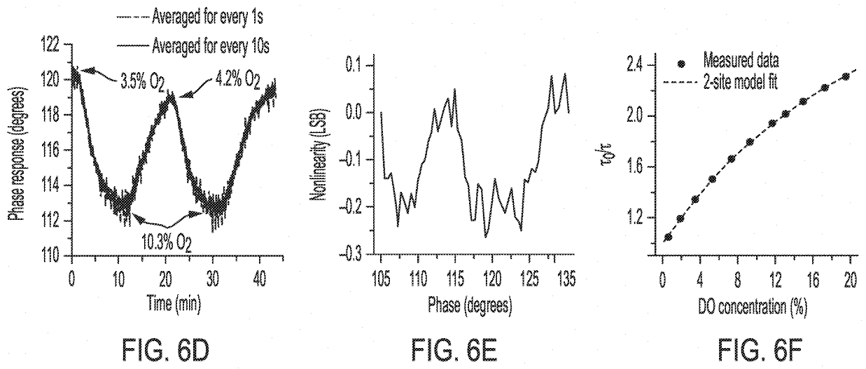

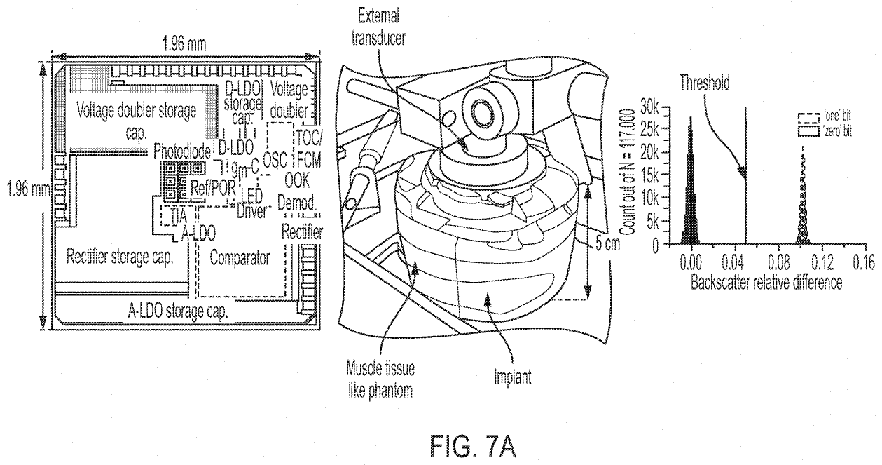

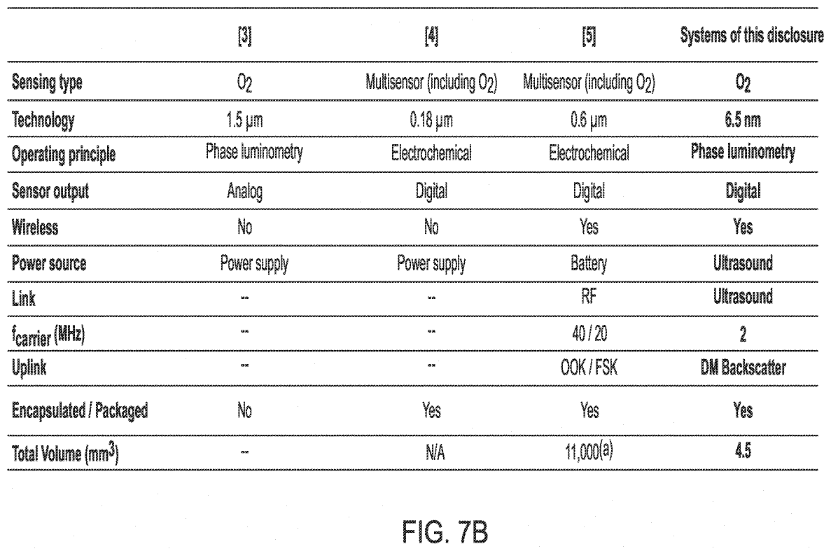

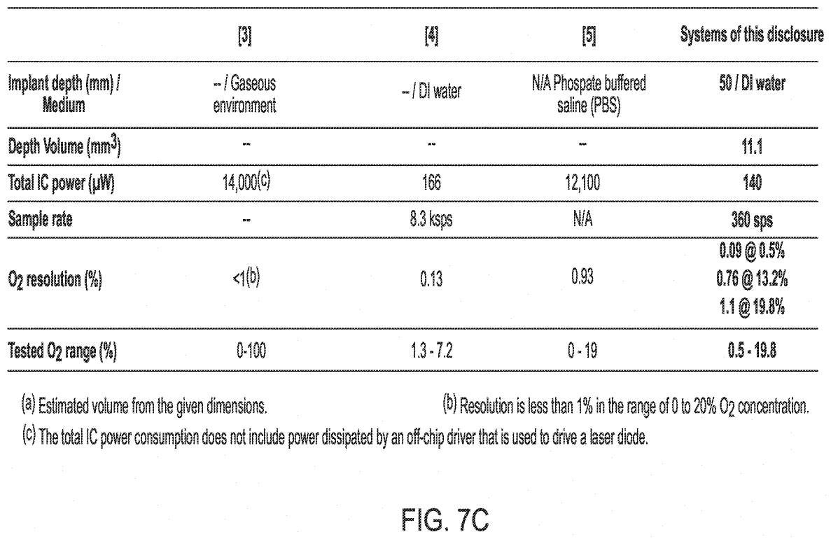

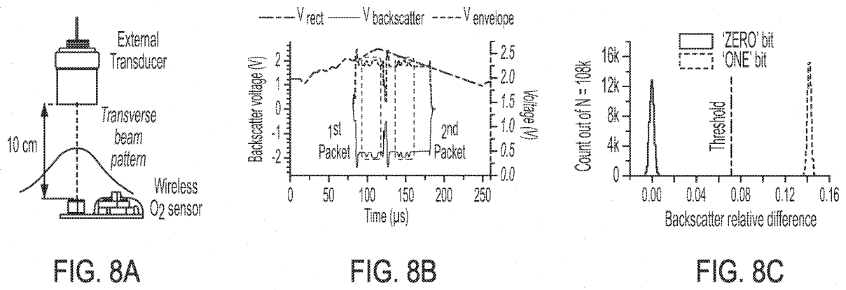

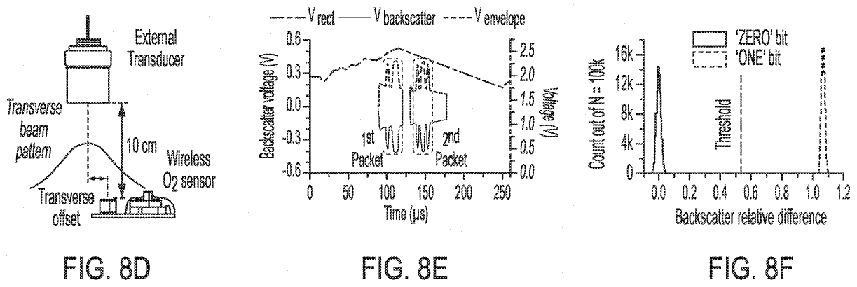

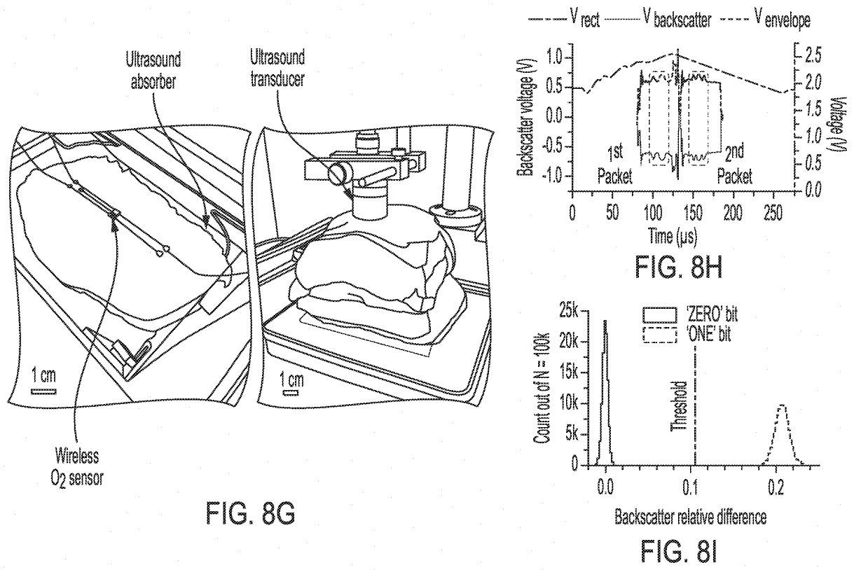

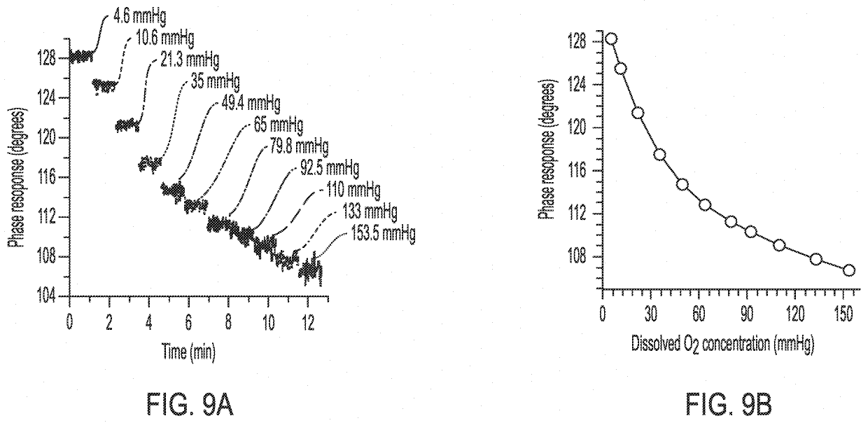

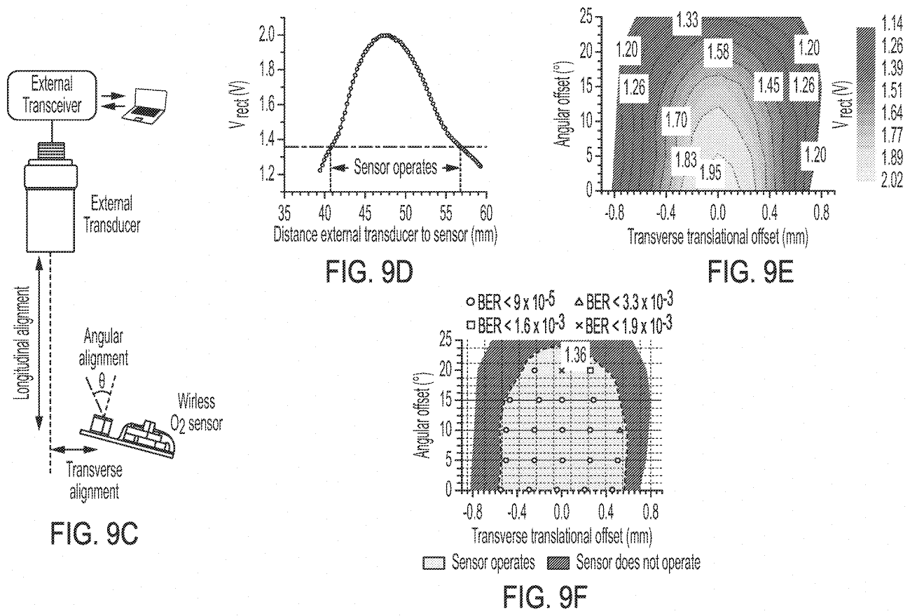

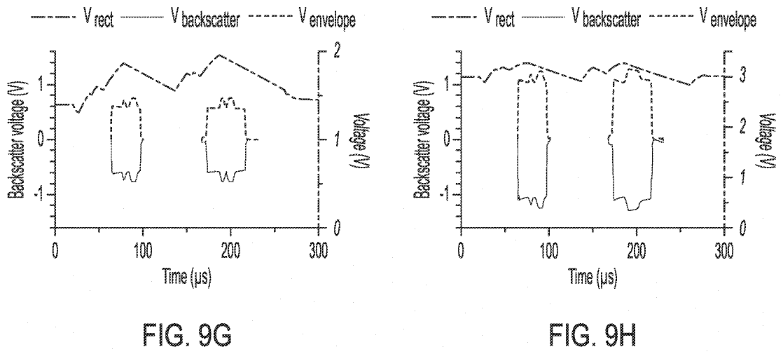

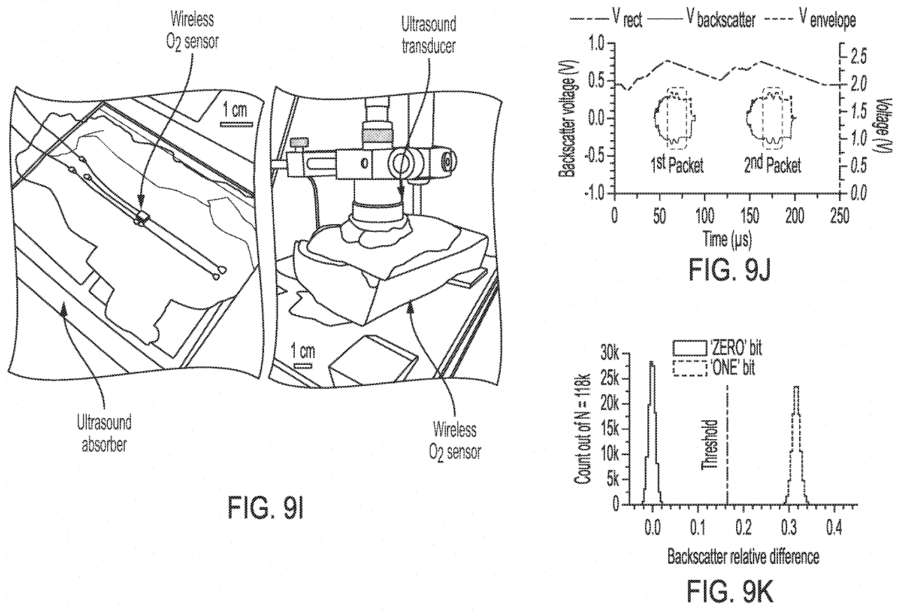

With further reference to the examples of FIGS. 1A-1J, these figures show an example biocompatible O₂-sensing film, operating principle of the luminescence O₂ sensor, and its optical characterization. FIG. 1C shows an expanded cross-sectional view of the luminescence O₂ sensor, and a model for the locus of Ru-dyes and O₂ molecules in silica-containing PDMS. The squares and circles represent the Ru-dyes and O₂ molecules, respectively (as indicated in FIG. 1C). The Ru-adsorbed silica particles were dispersed in PDMS. FIG. 1F shows normalized absorption and emission spectra of the Ru-dye in the O₂-sensing film along with normalized emission spectrum of the blue μLED and transmission spectrum of the optical filter. In some embodiments, the blue μLED, placed on the sensor platform to illuminate the O₂-sensing film, produces light with a peak intensity at ˜465 nm that excites the Ru-dyes in the O₂-sensing film. The excited Ru-dyes emit luminescence with a peak at ˜621 nm. A long-pass optical filter with a ˜550 nm cut-on wavelength suppresses excitation light and transmits luminescence, enabling the Ru-dye emission to be detected by an integrated circuit (IC) with an integrated photodiode. FIG. 1E is an Illustration of frequency-domain luminescence excitation and emission signals. FIG. 1G is a current-voltage-light output characteristics of the blue μLED. FIG. 1H illustrates a responsivity spectrum of an integrated photodiode with a 300×300 μm² active area and a reverse bias voltage of 0.6 V. FIG. 1H further illustrates a luminescence wavelength range, which is a wavelength range with strong luminescence emission from the excited Ru-dyes. FIG. 11 illustrates photobleaching of Ru-dye in the O₂-sensing film under continuous square-wave illumination with a peak excitation light power of ˜1.53 μW at the operating forward current of 24 μA, resulting in an average optical power density of ˜4.9 μW/mm² at the surface of the film, in air (21% O₂) at room temperature for a period of 60 h. FIG. 1J illustrates normalized luminescence intensity of Ru-dye in the PDMS film as a function of time after immersion of the same fully-packaged O₂ sensor used for the photobleaching test in PBS solution at 37° C. in room air. During the test, the sensor was operated with the same operating conditions as in the photobleaching test.

To further explain, and as discussed by in paragraphs 0131-0133 of WO 2018/009905, which is incorporated by reference herein, in some embodiments, an oxygen sensor comprises a Clark electrode. A Clark electrode measures oxygen on a catalytic surface (such as a platinum surface) surrounded by a membrane, and can be miniaturized to be included on an implantable device (e.g. a mote). The Clark electrode can be attached to an application-specific integrated circuit (ASIC) (e.g. a mote IC) on the implantable device, and variance in the amount of oxygen sensed by the implantable device (which may be blood oxygen or interstitial fluid oxygen) can modulate the ultrasonic backscatter.

In some embodiments, the oxygen sensor includes a light source (such as a light emitting diode or vertical cavity surface emitting laser (VCSEL)) and an optical detector (such as a phototransistor or a photovoltaic cell, or an array of phototransistors or photovoltaic cells). A matrix including an oxygen-sensitive fluorophore is disposed over the light source and the light detector, or in a position bridging the light source and the light detector, and the amount of light detected by the light source depends on the amount of oxygen in the surrounding fluid. Such devices can be referred to as optrodes. The matrix can include, for example, an oxygen-sensitive fluorophore (such as a ruthenium fluorophore), and increased oxygen (depending on the choice of fluorophore) can cause a faster decay of fluorescence and a decrease in intensity. This oxygen-dependent change in intensity and fluorescence decay lifetime can be detected by the optical detector. In some embodiments, the matrix is a hydrogel or polydimethylsiloxane (PDMS) polymer containing a ruthenium fluorophore. In some embodiments, the ruthenium fluorophore is bound to silica particles or silica surfaces contained within the matrix (these can be made by sol-gel processes, for example). The matrix protects the fluorophore from components in the extracellular fluid and inhibits adhesion of proteins, cells and other cellular debris that could affect the diffusion of oxygen into the matrix. Further, encapsulation of the ruthenium metal in the matrix reduces potential toxicity of the ruthenium. The light source and/or optical detector can optionally include a filter to limit emitted or detected light to a narrow bandwidth. The ASIC can drive the light source to emit a pulsed or sinusoidal light signal, which causes the light source to emit the light. The light emitted by the light source causes the fluorophore in the matrix to fluoresce. For example, in some embodiments, the light source emits a blue light or a UV light, and the fluorophore can emit an orange or red light. The fluorescence intensity and/or lifetime (decay) of fluorescence is a function of the oxygen concentration of the matrix, which is influenced by the surrounding fluid (e.g., blood or interstitial fluid). From the fluorescence decay, a fluorescent lifetime decay constant can be determined, which can reflects the oxygen amount.

Use of a light pulse emitted from the light source allows for the observation of fluoresce decay or fluorescence lifetime, which is dependent on oxygen concentration. Thus, in some embodiments, the decay of fluorescence (the fluorescence lifetime) following a light pulse from the light source is used to measure the oxygen concentration surrounding the sensor.

FIGS. 2A and 2B show an example overall system. An external piezo 220, driven by a high-voltage (HV) pulser 230, which includes both HV driver 240 and level shifter 250, sends US pulses into tissue when in transmit (TX) mode; these arrive at the mote 110 after one time-of-flight (ToF). In some embodiments, the external piezo 220 is located on the US interrogator. The mote 110 includes the mote piezo 120, and mote IC 130. The mote 110 uses the US pulses to power itself and to communicate back via amplitude-modulated (AM) backscatter pulses encoded in the ultrasound reflections from the mote's piezo. Reflected backscatter is received by the same external piezo 220 in receive (RX) mode, arriving at the mote 2 ToF after being sent during TX. The RX channel amplifies, filters, digitizes, demodulates and decodes the AM backscatter, providing real-time data. Interrogation via pulse-echo eliminates the need for a secondary external piezo for RX or a circulator. To avoid TX/RX overlaps, the duration of the AM backscatter pulses (TDM) must be set to <2 ToF, which limits the minimum distance (e.g. 2 cm) between the mote and the external piezo. To overcome this limitation, the digital O₂ data is sent via backscatter in two data packets (FIG. 3A).

With further reference to the example of FIG. 2A, an external transceiver is shown as including transmit (TX) and receive (RX) paths, where the TX path encoded downlink data onto a 2 MHz carrier. During TX operation, a level-shifter boosted a low-voltage transmit signal from a digital controller, and a high-voltage pulser drove an external piezo transducer 220. The RX path was enabled when the TX path was disabled. Reflected US backscatter from the sensor's piezo crystal was captured by the same external piezo transducer 220, which was digitized by the RX chain. The external piezo 220 coupled to the outside surface of tissue produced US waves traveling through tissue; these arrived at the sensor after one time-of-flight (ToF). The downlink provided power and a transmit command for the sensor. The uplink included of amplitude-modulated backscattered US waves that arrived at the external piezo 2 ToF after being sent during TX. FIG. 2B illustrates an example sensor IC architecture.

FIG. 2C shows another example schematic of the IC architecture, including more detail than the example of FIGS. 2A and 2B. In the example of FIG. 2C, an analog front-end (AFE) consisted of a transcapacitance amplifier, in which the DC feedback was provided using an active biasing circuit and the switches, controlled by ϕ_{TIA}, were implemented to minimize the settling time after duty-cycling, and of a comparator. The rectifier comparator outputs (Comp1 and Comp2) and the modulation signal (ϕ_{mod}) were used as inputs to the OOK demodulator that detected the envelope of the downlink signal and generated a notch. The TDC was based on a 10-bit synchronous counter and a phase detector. An LED driver was implemented using an 8-bit current digital-to-analog converter (DAC) that was driven by the 20 kHz 20 kHz complementary clock signals to avoid the rectifier voltage (V_{rect}) fluctuation. A cross-coupled voltage doubler was designed to boost V_{rect}. FIG. 3A shows an example timing diagram. The mote PZT starts harvesting power upon the arrival of an incident US pulse, which is rectified and regulated by the mote IC. When the low dropout (LDO) voltages are established, a power-on-reset (POR) signal is triggered to initialize the system. After a short initialization period (e.g., marked as the prepare transmission period/phase in FIG. 3A), the O₂ sensing operation begins, wherein Δϕ is converted to a 10-bit data that is divided into two 15 μs-long data packets with preambles; the first packet contains most significant bits (MSBs). The mote then listens for a falling edge in the data input from the interrogator; the notch prepares the mote for uplink transmission. Data packets are transmitted using digital backscatter modulation. To reduce energy consumption and hence eliminate the need for a large off-chip storage capacitor (e.g., a large Cₛₜₒᵣₑ), power-intensive blocks (e.g., front-end (e.g. as shown in FIG. 2B, which illustrates the front-end including the analog front end (AFE)), A-LDO, D-LDO, voltage doubler and/or LED driver) are duty-cycled off during uplink transmission. (In some embodiments, the power intensive blocks are duty cycled off during any phase illustrated by FIG. 3A that is not a receive backscatter phase; for example, the power intensive blocks are duty cycled off during any of the power up & sensing, prepare transmission, or data transmission phases.) Advantageously, in some implementations, this has allowed for a reduction of Cₛₜₒᵣₑ from 100 nF to 2.5 nF, which in turn allows for using, as Cₛₜₒᵣₑ, a capacitor with smaller physical dimensions and hence a smaller overall mote. In some embodiments, the uplink transmission stops if the notch duration is >64 μs, equivalent to ˜127 oscillations of a 2 MHz US carrier. The mote returns an O₂ sample after each such sequence; in this way, the sampling rate (f_{S}) can be externally controlled to reduce the mote and interrogator energy consumption.

In one example, the total area of the IC die, fabricated in a 65 nm low-power CMOS process, is ˜3.84 mm². In one example implementation, the minimum electrical input power required for proper operation of the IC was ˜150 μW, generating a rectifier voltage (V_{rect}) of ˜1.36 V, during the O₂-sensing phase. Power-intensive circuits (AFE, LED driver, voltage doubler, and TDC) were duty-cycled off during uplink transmission, reducing IC power consumption to ˜22 μW and thus avoiding the need for a large off-chip Cₛₜₒᵣₑ. The average power dissipation of the IC drops to less than 150 μW, including the rectifier's power conversion efficiency, during operation, depending on the O₂ sampling rate. The sampling rate (f_{S}) of the system was externally controlled through the external receiver.

In one example, during operation, the external transceiver was switched from TX to RX mode to capture uplink data encoded in the backscatter reflections from the sensor's piezo. The RX path demodulated and decoded the received backscatter, generating real-time O₂ data. The data was sent to a computer through a serial link for data storage and further analysis. In order to avoid overlapping TX and RX pulses, the data packet duration (TDM) was kept shorter than the round-trip time-of-flight (2ToF) of a US pulse between the sensor's piezo and the external transducer, (see, e.g., FIG. 2A), limiting the minimum operating distance between the sensor and the external transducer. In order to overcome this limitation, the digital data was divided into two parts that were subsequently sent to the external transceiver. FIG. 3B shows an alternative communication protocol for sensors implanted deeper than 5 cm because of the longer ToF to these depths. Compared to the protocol in FIG. 3A, the alternative protocol reduces the time spent during data transmission and hence increases the sampling.

It should be understood that in the examples of FIGS. 3A and 3B, during the power up & sensing phases, no data is transmitted. Thus, the power up & sensing phases in the example of FIGS. 3A and 3B are used only to power Cₛₜₒᵣₑ.

FIGS. 4A-4D show an example IC architecture. The power management circuits include an active full-wave rectifier 410 for AC-DC conversion, a voltage doubler 420 to boost the unregulated rectified voltage (V_{rect}) for driving the μLED, LDOs regulating supply at 1.2V for powering other circuits, and the biasing. The rectifier comparator outputs are used to drive the on-off keying (OOK) demodulator 430, detecting the downlink US envelope to generate a notch. The gm-C filter 440 generates V_{mid}, reducing noise on V_{ref,0.6}V. An LED driver 450 (8-bit current DAC) is designed to drive the LED 460 (e.g., a μLED) with a 20 kHz, 24 μA square-wave current. A replica driver 470, generating an opposite-phase current, is used to avoid V_{rect} fluctuation. The 300×300 μm² nwell/psub photodiode generates an emitted light-induced photocurrent (IPD). IPD is converted to a voltage by a transcapacitance amplifier, in which an active biasing circuit is used to provide the DC feedback and switches (ϕ_{TIA}) are used to minimize the settling time after duty-cycling. The amplifier output is compared to its DC component (V_{LPF,out}) by a comparator, performing zero-crossing detection, generating a time delay signal. A time-to-digital converter (TDC) is used to quantize the time delay into a digital representation of Δϕ. The digital output is serialized, divided, and transmitted through the PZT. The transistor switch (ϕ_{mod}) modulates the electrical load impedance (R_{L}) in shunt with the PZT series resistance (Rₚ), changing the acoustic reflection coefficient (Γ∝R_{L}/(R_{L}+R_{P})) at the PZT boundary and hence the backscatter amplitude.

In some embodiments, a time-to-digital converter (TDC), operating with a 16 MHz on-chip clock generated by a 5-stage current-starved ring oscillator, converted the time delay (phase difference, Δϕ) between the reference signal (ϕref), used to drive the μLED, and the luminescence signal (ϕPD) into a 10-bit digital data. The 10-bit data may be serialized and divided into two equal 15 μs-long data packets with a preamble and a postamble by a finite state machine; the first packet contains the most significant bits (MSBs). The measured minimum detectable average optical power, yielding a signal-to-noise ratio (SNR) equal to 1 at a 1 Hz bandwidth, was ˜1.3 pW at the peak emission wavelength of ˜621 nm and the operating frequency of 20 kHz for the optical readout, which was dominated by the noise of the AFE. The SNR was ˜53 dB under a typical ˜6.7 nW/mm2 light power after the optical filter, produced by the excited Ru-dyes in the O₂-sensing film, for the sensor operated in room air at 37° C. An uplink data transmission began when the on-off-keying (OOK) demodulator detected a “falling edge” in the US data input from the external transceiver, generating a notch (V_{OOK}). The notch served as a reference to time synchronize the sensor IC and the external transceiver during uplink transmission; a data packet was transmitted to the external transceiver after a notch with a duration of shorter than ˜64 μs, equivalent to 127 oscillations of a 2 MHz US carrier. Data packets were encoded in the US reflections from the sensor's piezo and transmitted via digital amplitude modulation of the backscatter. Backscatter amplitude modulation was achieved by modulating the electrical load impedance (R_{load}), in shunt with the piezo impedance (Zₚ), through a modulation (transistor) switch controlled by ϕ_{mod}, changing the US reflection coefficient at the piezo boundary, and thus the amplitude of the backscatter. When the transistor switch was turned on to transmit the O₂ data, the R_{load} across the piezo was reduced from a resistance value higher than 80 kΩ, depending on the IC power consumption and the amplitude of the rectifier input voltages, to ˜0.5 1Ω (transistor switch on-resistance). The uplink transmission stopped when the notch duration was kept longer than ˜64 μs.

In some embodiments, such as the example of FIGS. 4A-4D, there are two LDOs: a digital LDO (D-LDO) with an output of digital VDD (DVDD); and an analog LDO with an output of analogue VDD (AVDD). (see also the example of FIG. 3A, which shows AVDD and DVDD charged to a predetermined level of 1.2V).

FIGS. 5A and 5B show an example of an in vitro setup and measurements for an example mote operated at 50 mm depth using a 2 MHz acoustic wave with a 162 mW/cm² time-average intensity (˜23% of FDA limit). The first data packet consists of a ‘1’ preamble followed by 5-bit MSB, and the second consists of 5-bit LSB followed by a ‘1’ preamble.

In one example implementation, the system sampled oxygen 350 times per second with a resolution of <5.8 mmHg/√Hz across the physiologically relevant oxygen range of interest (0-100 mmHg) and a bit error rate of <10⁻⁵. The system was first characterized in a water tank setup (e.g., such as in FIG. 5A, etc.) where the distilled (DI) water temperature was kept constant at 37±0.1° C. to simulate physiological temperature, and O₂ concentration was monitored via a commercial O₂ probe and varied by controlling the ratio of O₂ and nitrogen (N₂) supplied to the water tank. Distilled water has an acoustic impedance similar to soft tissue (˜1.5 MRayls).

In one implementation, the example system was operated as described above, with an implant placed at a depth of 5 cm. Measured waveforms and backscatter signal were recorded for the system operated at a sampling rate (f_{S}) of 350 samples per second (Hz) using an acoustic field with a spatial-peak time-average intensity (I_{SPTA}) of 237 mW/cm² (˜32.9% of the FDA safety limit, ISPTA derated=720 mW/cm², for diagnostic ultrasound) (FIG. 5B). The system exhibited a power transfer efficiency from the acoustic power at the surface of the implant's piezo crystal to the electrical input power of the IC of ˜20.4% and a US link power transfer efficiency of ˜3.9%, defined as the ratio of the electrical input power of the IC to the acoustic power emitted from the external transducer. Each data packet was 15 μs long, containing 6-bits with 2.5 μs duration (e.g., FIG. 5B). The first data packet began with a ‘1’ preamble followed by 5-bit MSBs, and the second data packet began with 5-bit least significant bits (LSBs) followed by a ‘1’ postamble. The system achieved a modulation depth of ˜41%, and an uplink bit error rate (BER) less than 10⁻⁵ (0 out of 121k samples) with the optimal threshold, minimizing BER, determined by the external transceiver (FIG. 5C).

The system response to various O₂ concentrations and the Allan deviation of the data are illustrated in the examples of FIGS. 5D and 5E. An Allan deviation analysis was used to quantify the noise performance of the system. The system operated at 350 samples per second (f_{S}) exhibited an O₂ sensitivity greater than ˜0.066°/mmHg and a phase (ϕ) resolution less than 0.38°/√Hz, yielding an O₂-resolution better than 5.8 mmHg/√Hz across the relevant O₂ range of interest (<100 mmHg). Since the ϕ-resolution in the system is dominated by jitter noise of the AFE and the luminescence intensity (I) increases with a decreasing O₂ concentration, the system SNR, and hence ϕ-resolution, improves for lower O₂ levels. The nonlinearity of the phase readout circuit, including the photodiode, AFE, and TDC, was characterized using a function generator that produced a variable (ϕ)-shifted signal to drive the μLED. The worst-case nonlinearity, computed using the endpoint method, was less than 0.27 LSB=˜0.12° (see, e.g., FIG. 5L). The τ-based Stern-Volmer plot (see, e.g., FIG. 5P), obtained using the equation: Δϕ=tan⁻¹(2tfₒₚτ), reveals the nonlinearity at the system output, which is mainly due to heterogeneous dispersion of silica particles in the O₂-sensing film.

The luminescence O₂ sensor response to changes in O₂ concentration is reversible (see, e.g., FIG. 5I). Note that the example implementation used to generated the results of FIG. 5I used a calibration curve and equation to convert sensor phase output to O₂ concentration (partial pressure of oxygen, pO₂) in mmHg in this study (see FIG. 5Q). The response time (e.g., the time required to reach 90% of the steady-state value) of the sensor before black silicone encapsulation from ˜3.5 to ˜156 mmHg O₂ and vice versa were ˜210 s and ˜257 s, respectively; this increased to ˜250 s and ˜320 s after black silicone encapsulation of the O₂ sensor.

In order to assess the potential effect of sterilization on the O₂ sensor functionality, the sensors were first sterilized in ethylene oxide (EtO), and then their response to O₂ changes was tested in DI water at 37° C. The sensor response to O₂ variation before and after sterilization was nearly identical (FIG. 5J), indicating that these sensors are sterilizable without loss of their functionality.

To evaluate the ability of the O₂ sensor to resist in vivo biofouling, in vitro experiments were conducted in phosphate-buffered saline (PBS, 1×) and undiluted pooled human serum. Note that human serum, a complex fluid containing hundreds of different proteins, was chosen to simulate in vivo fouling since nonspecific protein adsorption (fouling) on the implant surface is considered the initial step in triggering a foreign-body reaction and one of the critical factors causing failure of many implants. The response of the O₂ sensors incubated in PBS and serum at 37° C. was measured at low (6.5 mmHg) and high (156 mmHg) oxygen levels and different times during 10-days (FIG. 5k). The sensors demonstrated no apparent loss of sensitivity to O₂ changes during these 10-days.

In one example implementation, FIG. 5C shows backscatter relative difference for 121k O₂ samples, showing ˜41% modulation depth. To detect each bit that is either “0” or “1”, the amplitude of the backscatter signal in the data packets was compared to a threshold. The wireless system achieved an uplink bit error rate (BER) less than 10⁻⁵ (0 out of 121k samples) in the measurement, demonstrating a robust data uplink. In another example implementation, FIG. 5E shows Allan Deviation of the raw data (shown in FIG. 5F). FIGS. 5I and 5J show O₂ sensor response to changes in O₂ concentration in DI water at 37° C. before and after black silicone encapsulation (FIG. 5I) and ethylene oxide (EtO) sterilization (FIG. 5J). FIG. 5K shows data from the O₂ sensors incubated in PBS and undiluted human serum at 37° C. for 10 days.

Another example implementation tested the clinical utility of the wireless, direct O₂ monitoring system in a physiologically relevant large animal model (a sheep). The sheep model is a standard in fetal, neonatal and adult disease states due to the remarkable similarities in cardiovascular and pulmonary physiology, neurobiology, as well as metabolism. Anesthetized juvenile or adult sheep (n==2 animals) were intubated and mechanically ventilated. The biceps femoris was carefully dissected and the wireless sensor, as well as a commercial wired pO₂ sensor, were placed in the plane below the muscle layer; and the muscle, as well as overlying skin, were closed above. The ultrasound transducer, attached to a five-axis micromanipulator for fine alignment, was placed on top of the skin layer on an acoustic standoff pad.

As seen from the data (from animal A) in FIG. 5N, and determined by the wired pO₂ sensor, acutely adjusting the inspired O₂ concentration from 100% to 10% resulted in a rapid reduction in muscle pO₂ from approximately 90 mmHg to around 20 mmHg. The wireless sensor similarly was able to accurately reflect muscle pO₂ levels.

More gradual, stepwise reductions in inspired oxygen content resulted in more gradual reductions in tissue pO₂, which were accurately determined by a wireless sensor in real-time with similar kinetics as the commercial wired probe (FIG. 5O; the data from animal B). Notably, simultaneous determination of blood hemoglobin saturation via pulse oximetry was not capable of detecting differences in tissue or blood oxygenation above room air (21%), as all hemoglobin is fully saturated beyond this point. In sum, and as will be seen, the exemplary mm-scale wireless implantable oxygen sensor accurately reflects tissue oxygenation status under physiological states and therefore has significant potential to augment clinical decision making in settings where tissue or patient oxygenation status warrants careful monitoring.

Regarding the example of FIG. 5N, the anesthetized animal was provided with 100% inspired oxygen via an endotracheal tube, followed by a hypoxic gas mixture of 10% O₂ achieved via nitrogen blending and confirmation with an inline O₂ detector, followed by ventilation with room air (21% O₂). Tissue O₂ concentration readings were continuously monitored via the wireless O₂ sensor as well as the wired commercial NEOFOX probe. Corresponding paO₂, SpO₂ and FiO₂ readings are provided. The example of FIG. 5O illustrates a stepwise reduction in FiO₂ which resulted in corresponding stepwise reductions in tissue pO₂ readings that showed excellent concordance between the wireless O₂ sensor and the commercial probe. Corresponding paO₂, SpO₂ and FiO₂ readings are provided. Note that for FiO₂ values above room air, SpO₂ readings are unable to approximate paO₂ or tissue pO₂ levels.

In another example, to further evaluate uplink performance, the system was operated at 350 Hz f_{S} and 10 cm depth in DI water with and without intentional misalignment using the same water tank setup (FIGS. 8A and 8D) and through fresh, ex vivo porcine tissue (which presented inhomogeneous acoustic properties) (FIG. 8G). The system, while operating without any misalignment and using a 2 MHz acoustic field with an I_{SPTA} of 282 mW/cm², exhibited a modulation depth of over 14%, a BER lower than 10⁻⁵ and a US link power transfer efficiency of ˜0.74% (FIGS. 8A-C). When operated with an intentional transverse offset of ˜1.21 mm between the central axes of the acoustic field and the sensor piezo, the system demonstrated a modulation depth of ˜108% and a BER lower than 10⁻⁵, but at the expense of a ˜58.9% increase in I_{SPTA}, resulting in a concomitant reduction in the US link power transfer efficiency (FIGS. 8D-F). As seen from the amplitude of the unmodulated backscatter signals (FIGS. 8B and 8E), the increase in modulation depth was mainly due to reduced reflections from non-responsive regions (here, the sensor surface), indicating that an acoustic field with a smaller transverse beam spot size (e.g. comparable to the piezo size) can provide more robust uplink performance and higher US link power transfer efficiency.

In this example ex vivo measurement, the external transducer generated US waves with 706 mW/cm² I_{SPTA} de-rated by 0.3 dB·cm⁻¹·MHz⁻¹ (FDA-standard US attenuation in soft tissue²⁴), producing an acoustic power of ˜228 mW at the transducer surface, that propagated through approximately 3 mm ultrasound gel, 1 mm skin, 1 mm fat, and 95 mm muscle tissue (FIG. 8G). The system achieved ˜21% modulation depth, a BER of <10⁻⁵, demonstrating a robust uplink performance, and a US link power transfer efficiency of ˜0.08% with the sensor consuming ˜194 μW average electrical power (FIGS. 8E and 8F). Note that uplink performance (modulation depth and BER) of the system depends on acoustic attenuation due to scattering and absorption in heterogeneous tissue, which varies for different tissue types/specimens. This is because acoustic absorption and dispersion may change the amplitude of the unmodulated backscatter signal received by the external transceiver during the time interval within the uplink data is received. Furthermore, US link power transfer efficiency is also a function of acoustic attenuation; for example, the system operated through a porcine tissue specimen (FIG. 8G) exhibited a significantly lower link power transfer efficiency than the system operated in distilled water (FIG. 8A) since the US attenuation of a tissue sample is much higher than that of water.

FIGS. 8A-8I illustrate examples relating to in vitro and ex vivo uplink characterization of an exemplary wireless oxygen-sensing system. The exemplary systems of FIGS. 8A and 8D both include wireless sensor operation at 10 cm depth in distilled water. However, the example of FIG. 8A does not include an intentional transverse offset of the central axis of the acoustic field to that of the sensor's piezo; whereas, the example of FIG. 8D does include this intentional transverse offset. FIGS. 8B and 8E illustrate examples of sensor waveform and backscatter signal captured in the wireless measurement of a single O₂ sample for the sensor operated in vitro at 10 cm depth without a transverse misalignment (FIG. 8B), and with a transverse misalignment (FIG. 8E). FIGS. 8C and 8F illustrate backscatter relative difference for wirelessly recorded O₂ samples ≥100k from the sensor operated in vitro at 10 cm depth without (FIG. 8C) and with (FIG. 8F) a transverse misalignment. FIG. 8G illustrates an example where the O₂ sensor was wirelessly operated at a depth of 10 cm through an inhomogeneous sample of fresh, ex vivo porcine tissue, in which acoustic waves passed through approximately 3 mm ultrasound gel, 1 mm skin, 1 mm fat, and 97 mm muscle tissue. FIG. 8H illustrates an example where the sensor waveform and backscatter signal were recorded in the wireless measurement of a single O₂ sample for the sensor operated at 10 cm depth through a porcine specimen. FIG. 8I illustrates a backscatter relative difference for 100k O₂ samples, showing ˜21% modulation depth, from the sensor operated at 10 cm depth through a porcine tissue specimen. The system achieved an uplink BER of <10⁻⁵ in the measurement.

### DISCUSSION OF SYSTEM AND EXAMPLES

The following presents the first miniaturization of a fully implantable optrode in the mm³ volume range, which is suitable for deep-tissue measurements. Although the focus of the following is a system for measuring oxygen tension in vivo, the fundamental technological achievement opens the door to minimally invasive pulse oximetric sensors, pH sensors, CO₂ sensors among others. Each of these, embodied in ultra-miniature and deep tissue systems, would open the door to novel diagnostics.

With regard to the measurement of deep-tissue oxygen tension, the application space of the wireless O₂-sensing system is vast. Organ transplantation provides a clear example. The demand for organ transplantation continues to grow. In 2019, there were 94,863 candidates on the waiting list for renal transplants in the U.S. alone. [see National Data—OPTN. https://optn.transplant.hrsa.gov/data/view-data-reports/national-data/]. Monitoring graft oxygenation following organ transplantation is critical but typically relies on indirect methods that require skilled operators and provide only intermittent snapshots of tissue perfusion. Continuous and reliable monitoring of graft oxygenation following orthotopic liver transplantation, for example, may enable early detection of graft ischemia due either to hepatic artery thrombosis or graft vascular disease, allowing timely surgical re-exploration to minimize risk of graft loss, which could be fatal. Notably, these complications can occur months to years following transplant. Minimally invasive wireless modalities, such as those described herein, could enable real-time monitoring of graft oxygenation via wearable applications in the out of hospital setting, providing critical information regarding tissue oxygenation before the emergence of graft dysfunction, allowing timely intervention. This would additionally help differentiate parenchymal rejection from graft vascular disease when organ dysfunction emerges. Furthermore, more than 5.7 million patients are admitted annually to intensive care units (ICUs) in the United States (U.S.). Assessment of tissue oxygenation is a fundamental need in this setting. Although some embodiments require surgical placement, some contemplated embodiments may enable semi-invasive/vascular approaches for probe placement. Depending upon the underlying pathology, the local oxygen supply-demand balance can be distorted during pathological states, such as observed during various forms of shock. Thus, an inadequate delivery—for whatever reason—relative to demand will decrease tissue pO₂. On the other hand, a primary reduction in metabolic demand or an inhibition or failure of mitochondrial oxidative phosphorylation will leave oxygen supply largely unaffected, and thus, the tissue pO₂ may increase. A close matching of oxygen supply and demand, be it via an overall increase in delivery or decrease in turnover, will result in no net change in tissue pO₂. Global measures of cardio-pulmonary performance such as cardiac output, oxygen delivery or blood pressure frequently do not reflect local metabolic demands at the organ and tissue level and can promote excessive fluid loading or inotrope dosing, worsening outcomes. A notable contributor in this setting is a lack of hemodynamic coherence between the microcirculation and the macrocirculation. Given that these changes typically occur over minutes to hours, a slightly longer response time than typically observed for pulse oximetry would still yield important clinical information. Coupling direct measurements of the microcirculation with direct monitoring of tissue pO₂ would greatly augment critical care management approaches. Precise measurements of tissue oxygen content are therefore instrumental for the proper management of shock states, but are currently limited to indirect or surface deep methods. Novel non-invasive as well as minimally invasive modalities for monitoring deep-tissue oxygenation, as described herein, are clearly needed to advance our understanding and management of disease states where oxygen delivery or metabolism is compromised.

To clinically adopt the wireless O₂-sensing system for the use of chronic, real-time in vivo O₂ tracking, a number of technical challenges must be addressed. One of the main challenges is the post-surgical localization of the implant by an external transceiver since the post-surgical in vivo position may drift relative to any external fiducials; such movement can arise due to pressure from outside the body, movements or breathing of the subject, and scar formation. The in vivo localization before each pO₂ measurement can be achieved with an external phased-array transceiver that utilizes ultrasound (US) backscatter information first to find and then track the time-dependent position of the implant in the body.

A second challenge arises because acoustic attenuation due to scattering and absorption varies between different US propagation paths to the implant in heterogeneous tissue; a path with higher attenuation in tissue may significantly degrade power transfer efficiency and data transfer reliability of the system. For example, muscle tissue with a more unevenly distributed intramuscular fat content will exhibit greater acoustic attenuation. Here too, an external transceiver with a large-aperture, multi-element transducer array capable of focusing US energy to the implant will allow for steering the US beam along a preferred path. Finally, a phased array could also potentially be used to interrogate multiple O₂ sensors implanted in different locations of target tissue in a time-division multiplexing fashion or simultaneously.

In addition to these improvements, chronic in vivo use of the wireless O₂ sensor will require hermetic packaging to prevent biofluid penetration into the electronic sensor components (IC and μLED) and the piezo crystal. Traditionally such millimeter-scale implantable hermetic housings make use of ceramic or titanium enclosures brazed or microwelded to achieve the required hermeticity. This is an active area of work both commercially and academically; an extensive review was recently published. [Shen, K. & Maharbiz, M. M. Ceramic Packaging in Neural Implants. bioRxiv 2020.06.26.174144 (2020) doi:10.1101/2020.06.26.174144]. Acoustic windows for efficient ultrasonic energy transfer into ceramic or metallic housings have recently been demonstrated in the academic literature. [Shen, K. & Maharbiz, M. M. Design of Ceramic Packages for Ultrasonically Coupled Implantable Medical Devices. IEEE Trans. Biomed. Eng. 67, 2230-2240 (2020)]. Some embodiments described herein use biocompatible polymer materials (parylene-C, silicone and UV-curable epoxy) to encapsulate the sensor given their ease of use for acute and semi-chronic experiments. It should be noted that polymeric materials at these thicknesses are not suitable for long term in vivo use of the implant due to their high water vapor permeability.

### EXAMPLE METHODS AND EXPERIMENTAL DATA

### Fabrication of an Example Oxygen-Sensing Film

Some implementations of the film fabrication included two steps. First, luminescent dyes, tris-(Bathophenanthroline) Ruthenium (II) Perchlorate (Ru(dpp)₃(ClO₄)₂) (CAS 75213-31-9; GFS Chemicals), were immobilized on the surface of silica particles with a diameter of 10 μm (CAS 7631-86-9; LiChrosorb Si 100 (10 μm); Sigma-Aldrich) at about 1:10 dye:particle ratio by weight. Briefly, 200 mg Ru(dpp)₃(ClO₄)₂ complex was dissolved in 10 ml ethanol (ACS reagent ≥99.5%, CAS 459844; Sigma-Aldrich). Silica gel was prepared by adding 2 g silica particles to 40 ml aqueous NaOH (0.01 N; CAS 1310-73-2; Fisher Scientific) solution and magnetically stirring the mixture at a speed of 1000 rpm for 30 min. Next, the dye-containing ethanol solution was poured into the silica gel solution and stirred at 1000 rpm for 30 min. The dye-containing silica particles were filtered out of the solution through a filter with a pore size of 0.45 μm (Catalog number 165-0045; ThermoFisher Scientific), and then washed once in ethanol and three times in deionized water. All the supernatant was removed, and the dye-loaded silica particles were dried at 70° C. overnight.

Second, the dye-loaded silica particles were incorporated into polydimethylsiloxane (PDMS) to avoid problems related to dye leaching in aqueous media. 2 g dried silica particles were thoroughly mixed with 20 g PDMS prepolymer Part A and 2 g PDMS curing agent Part B (Sylgard 184; Dow Corning). A ˜100 μm-thick film was prepared by spinning a small amount of this mixture at 500 rpm on a microscope slide and then by curing it at 60° C. under dark and vacuum (<10 Torr) for ˜7 days, to remove solvent and air bubbles. The cured film was kept under dark at room temperature for at least 24 h before use and stored under dark at room temperature.

### Design, Fabrication, and Assembly of an Example Wireless Oxygen Sensor

In some implementations, the wireless sensor was built on a 100 μm-thick polyimide, flexible PCB with electroless nickel immersion gold (ENIG) coating (Rigiflex Technology). A 750 μm-thick lead zirconate titanate (PZT) sheet with a 12 μm-thick fired on silver electrodes was diced using a dicing saw with a 300 μm-thick ceramic-cutting blade. A 750 μm³ PZT cube was first attached to a flexible PCB using two-part conductive silver epoxy with 1:1 mix ratio (8331, MG Chemicals), and then the board was cured at 65° C. for 15 min, well below the PZT Curie temperature and the melting temperature of polyimide. The top electrode of the PZT was wire bonded to the PCB using a wedge bonder (747677E; West Bond) to create an electrical connection between the PZT and the IC. The board was then encapsulated with ˜10 μm-thick layer of parylene-C using chemical vapour deposition (Specialty Coating Systems) for insulation due to its biological inertness and resistance to a moisture. The ˜10 μm-thick Parylene-C reduces the power harvesting efficiency of the PZT by ˜49% by damping its vibrations. The metal pads on the PCB for the IC and its wire bonds were carefully exposed by scoring the parylene around the pads using a sharp probe-tip and removing the parylene layer. The IC was attached to the PCB using the same silver epoxy, cured at 65° C. for 15 min, and then wire bonded to the PCB. Next, a ˜250 μm-thick optical long-pass filter with a cut-on wavelength of 550 nm (Edmund optics) were attached to the top of the IC using medical-grade, UV-curable epoxy (OG142; Epotek). The same UV curable epoxy was also used to assemble other sensor components, including a μLED with dimensions of 650 μm×350 μm×200 μm (APG0603PBC; Kingbright) and its 3D-printed holder (Protolabs), to protect the wire bonds of the chip and μLED and provide insulation. After the assembly was completed, the ˜100 μm-thick O₂-sensing film was slipped through the gap between the μLED holder and the optical filter. The small residual space between the μLED holder and the film was filled by PDMS (Sylgard 184; Dow Corning). PDMS Sylgard 184 Part A and B were mixed in the ratio of 10:1, degassed, poured between the space, and cured at room temperature for 48 h. Finally, the oxygen-sensing region on the IC was coated with a ˜180 μm-thick layer of biocompatible, highly O₂-permeable black silicone. The black silicone consisted of two-part, low-viscosity silicone elastomer (MED4-4220, NuSil Technology, LLC) and black, single component masterbatch (Med-4900-2 NuSil Technology, LLC); the two silicone parts (A and B) were first mixed in a 1:1 weight ratio, and then the masterbatch (4% by weight) was added, thoroughly mixed, degassed for <˜5 min, applied to the sensor surface, and cured at room temperature for 48 h.

In this example, PZT was selected as a piezoelectric material due to its high electromechanical coupling coefficient and high mechanical quality factor, providing high power harvesting efficiency. A lead-free biocompatible barium titanate (BaTiO₃) ceramic with a slightly lower electromechanical coupling coefficient can be used in place of PZT.

The volume of a wireless O₂ sensor was measured by using a suspension technique. In volume measurements, the sensor without test leads was suspended with a thin, rigid wire below the water surface in a container placed on an electronic balance with a measurement accuracy of 0.1 mg. The volume of the sensor was calculated from the weight difference of a water-filled container before and after submersion of the sensor in water; the weight difference, equal to the buoyant force, was divided by the density of water to determine the actual sensor volume. The volume measurements were performed using two separate sensors; the volume of each sensor was measured five times to determine reproducibility. The data obtained from all the volume measurements were presented by the mean and standard deviation values (mean±2s.d.).

### Optical Characterization of Components of the Luminescence Oxygen Sensor

In some implementations, the absorption spectrum of the O₂-sensing film and the transmission spectrum of the optical filter were measured with a Jenway 6300 spectrophotometer. The emission spectra of the sensing film and the blue μLED was measured using a fiber-coupled CCD spectrometer (Thorlabs, CCS200/M) operating at an integration time of 1 s and enabled with electric dark correction. The film samples were excited at 450 nm by a laser diode (Osram, PL450B, purchased from Thorlabs) driven with a Keithley 2400 source meter, and its emission was scanned in the range of 515-800 nm. The optical output power level of the μLED was measured using an optical power meter (Thorlabs, PM100D) equipped with a Si photodiode detector (Thorlabs, S121C). The current-voltage curve of the μLED was measured with a Keithley 2400 source meter. The responsivity of the photodiode as a function of wavelength was measured using a halogen lamp coupled to a monochromator, a reference photodiode (Thorlabs, FD11A Si photodiode) and an Agilent B2912A source meter. The same photodiode (FD11A) was also used to measure the output light intensity of the μLED.

### Photobleaching and Leaching Tests

Photobleaching of the Ru-dye in the O₂-sensing film was evaluated using a fully-packaged O₂ sensor (FIG. 1B) continuously operated in room air (21% O₂) at room temperature for 60 h period. After the photobleaching test, the same sensor was continuously operated in phosphate-buffered saline (PBS, 1×) without calcium and magnesium (Corning; Mediatech Inc.) at 37±0.1° C. in an oven (Test Equity Model 107) for 14 days, to assess leaching and further photobleaching of the Ru-dye in the film. In the tests, the sensor was electrically driven by differential, 2 MHz AC signals from a Keysight 33500B function generator, which are ac-coupled to the rectifier inputs of the IC. The output of the transimpedance amplifier (TIA) (FIG. 2C) was connected to a buffer (LTC6268; Linear Technology). During the tests, the buffer output at the excitation frequency of 20 kHz was continuously measured using a 14-bit digitizer (NI PXIe-5122; National Instruments) with a sampling rate of 2 MHz. A custom Labview program (Labview 2018; National Instruments) was developed to detect and record the peak-to-peak amplitude of the buffer output that is directly proportional to the luminesce intensity of Ru-dye immobilized in the O₂-sensing film. The collected data were averaged every 3 and 12 hours in FIGS. 1I and 1J, respectively.

### Design of an Example External Transceiver

The external transceiver consisted of transmitter (TX) and receiver (RX) paths. The TX path included a commercial high-voltage pulser with an integrated TX/RX switch (MAX14808; Maxim Integrated) and a digital controller module (NI PXIe-6363; National Instruments). During the TX mode, the high-voltage pulser converted a low-voltage signal from the digital controller module to a high-voltage signal, necessary to drive an external ultrasound transducer to generate ultrasound pulses. The RX path included an ultralow noise amplifier (AD8432; Analog Devices) to receive and amplify the backscatter signal from the external transducer, a gain amplifier to further amplify the signal to a level within the input range the analog-digital converter (ADC), and a digitizer with an antialiasing filter and a 14-bit high-speed ADC (NI PXIe-5122; National Instruments) to filter and digitize the signal after receiving and amplification. In addition to the switch integrated into the pulser, an extra digitally-controlled switch (ADG619; Analog Devices) was used to minimize the electrical coupling (interaction) between the TX and RX paths. The TX and RX paths were synchronized to each other by using the same reference clock integrated into the backplane of the PXI chassis (NI PXIe-10620; National Instruments). Note that the digital controller, digitizer, and NI PXIe-8360 modules were inserted in the chassis, in which the NI PXIe-8360 module was used to connect the chassis to a computer for communication with the other modules and data transfer.

A custom Labview program (Labview 2018; National Instruments) was developed to control the modules and to process the backscatter data in real-time. A (TX and RX) communication protocol was encoded in the program. During real-time data processing, the backscatter data digitized by a 14-bit ADC with a sampling rate of 20 MHz were resampled by a factor of five and then interpolated with a sinc function. The sinc interpolation was followed by a peak detection to extract the envelope of the backscatter signal and linear interpolation to increase the number of data points and hence to improve the accuracy in the determination of an optimal threshold value that minimizes bit error rate (BER). An optimal threshold (that is, the half value of the sum of modulated and unmodulated backscatter signal amplitudes) was determined by taking the mean of the data points from the time intervals where the steady-state backscatter signal was amplitude modulated and unmodulated. The threshold was used to convert the digitized data into digital format: bits (“0” or “1”). The bits were scanned to find a preamble and a postamble and hence to extract data bits. The binary coded data (bits) were converted to numeric data, which was stored on a computer.

### Example In Vitro and Ex Vivo Characterization

An in vitro characterization of the wireless oxygen monitoring system was performed in a custom-built water tank using a 25.4 mm diameter, 2.25 MHz single-element external ultrasonic transducer (V304-SU-F1.88IN-PTF; Olympus) with a focal depth of 47.8 mm, mounted on manual translation stages (Thorlabs) and connected to an external transceiver board, at various alignments and positions of the wireless oxygen sensor with test leads, mounted on top of a steel rod with a diameter of 0.75 mm connected to a manual rotation stage (Thorlabs). In measurements, the external transducer face was covered with a thin sheet of latex by filling the empty space between the transducer face and the latex sheet with castor oil (used as a coupling medium), to protect the matching layer of the transducer from possible damage due to the long-time direct contact with water or ultrasound gel. A hydrophone (HGL-0400; Onda) was used to calibrate the output pressure and hence the acoustic intensity and to characterize the acoustic beam patterns of the external transducer (FIGS. 5T and 5U).

In measurements, the water tank was placed on a stirring hotplate (Thermo Scientific Cimarec), to keep the water temperature constant at 37±0.1° C., to simulate physiological temperature, and to stir using a magnetic stirrer to increase the speed of a transition from low to high O₂ level and vice versa in distilled water. Water O₂ concentration was monitored using a commercial O₂ probe with a 300 μm core diameter (NEOFOX-KIT-PROBE; BIFBORO-300-2; Ocean Optics) varied by controlling the ratio of O₂ and N₂, supplied to the water tank through two pipes, via a matched pair of gas flow controllers (FMA-A2407; Omega) connected to O₂ and N₂ gas cylinders. A customized Matlab program controlled the gas flow controllers through a digital-to-analog converter board (NI myDAQ; National instruments) connected to a computer.

The measured phase output data from the wireless system were converted to O₂ concentration (partial pressure of oxygen, pO₂) in mmHg by an exponential equation: pO₂ (mmHg)=A·e^{(B/(ϕ)}+C, where ϕ is the phase output, and A, B and C are constant coefficients obtained from curve fitting (see FIG. 5Q).

Ethylene oxide (EtO) sterilization with an exposure time of 4 h at 37±3° C. and an aeration time of 24 h at 37±3 ° C. was performed by a commercial vendor (Blue Line Sterilization Services LLC, Novato, Calif.).

To assess the functionality of the fully-packaged O₂ sensors (FIG. 1B) over time in an environment that mimics (to first order) in vivo biofouling, phosphate-buffered saline (PBS, 1×) without calcium and magnesium (Corning; Mediatech Inc.) and pooled human serum (off the clot) (purchased from Innovative Research, Inc., Novi, Mich.) were used for 10-day incubation of the sensors at 37±0.1° C. in an oven (Test Equity Model 107). The two antibiotics, penicillin and streptomycin with a final concentration of 100 units/mL and 100 μg/mL (Gibco by Life Technologies, Catalog #15-140-122; purchased from ThermoFisher Scientific), were added to human serum to inhibit bacterial growth during the study. The test in serum was performed by placing the sensor in a container, where antibiotics-added serum was replaced every 24 h to ensure sterile conditions during the length of the study. In the fouling tests, the sensors were operated at 350 samples per second (Hz) sampling rate with differential, 2 MHz AC signals produced by a Keysight 33500B function generator, which are ac-coupled to the rectifier inputs of the IC. One of the rectifier inputs was connected to a high-input impedance buffer amplifier (LTC6268; Linear Technology). The buffer output, O₂ data, was recorded by a 14-bit high-speed digitizer (NI PXIe-5122; National Instruments), synchronized to the clock of the function generator, and a custom Labview program (Labview 2018; National Instruments).

Measurements for uplink performance assessment of the system at 10 cm depth in DI water and through a fresh ex vivo porcine specimen were performed with a custom-designed and -built spherically-focused, 2 MHz, 25.4 mm diameter ultrasonic transducer with a focal length of 88.1 mm (Sensor Networks Inc.) (see FIGS. 5V and 5W for the transducer beam pattern). In the ex vivo measurements, a porcine tissue sample was positioned between the wireless sensor and the external transducer, with coupling enabled by ultrasound gel (Aquasonic Clear; Parker Labs). Air bubbles in ultrasound gel were removed via centrifugation at 2800 rpm for 10 min. In order to remove possible air bubbles entrapped between the sensor and the tissue, the sensor was positioned on a tissue sample in a container filled with DI water. A piece of ultrasound absorbing material was placed under the tissue sample to avoid ultrasound reflection from the bottom interface of the container.

### Backscatter Modulation Depth

Backscatter relative difference is defined as the ratio of the amplitude difference between the modulated and unmodulated backscatter signals to the amplitude of the unmodulated backscatter signal. The modulation depth percentage was calculated by multiplying the backscatter relative difference by 100. Backscatter relative difference plots were obtained by collecting data samples from the time points where the steady-state backscatter signal was amplitude modulated and unmodulated during the O₂ measurement.

### Ultrasound (US) Link Power Transfer Efficiency

The US link power transfer efficiency is defined as the ratio of the electrical input power of the IC to the acoustic power emitted from the external transducer, which depends on the beam focusing ability of the external transducer, the frequency-dependent attenuation of US intensity in the propagation media, and the power conversion efficiency of the sensor. The acoustic power at the transducer surface was calculated by integrating the acoustic field intensity data, obtained by a hydrophone at the focal length, over a circular area where the intensity of the side lobes is not negligible. The power conversion efficiency of the sensor, relying on the receive (acoustic-to-electrical conversion) efficiency of the piezo and the impedance matching between the piezo and the IC, is equal to the ratio of the electrical input power of the IC to the acoustic power at the surface of the sensor piezo; the acoustic power at the piezo surface was calculated by integrating the acoustic field intensity data from the hydrophone over the surface of the sensor piezo.

### In Vivo Measurements

Tissue pO₂ measurements were performed with the wireless system operated at a sampling rate of 350 samples per second. In the in vivo measurements, the maximum distance from the external transducer to the wireless O₂ sensor, operated with an acoustic field that had a derated ISPTA of 454 mW/cm² and a mechanical index of 0.08 (both below the FDA regulatory limits of 720 mW/cm² and 0.19), was ˜26 mm with ˜19 mm consisting of tissue (including skin, fat, and muscle). The distance between the implanted sensor and the external transducer was estimated from the round-trip time-of-flight (that is, the time delay between the received backscatter signal from the sensor piezo and the signal that drove the external transducer). Both the wireless and the wired data, were averaged every 5 s. Two identical wireless O₂ sensors were used in in vivo measurements; the first sensor response to various O₂ concentrations in water and animal A was shown in FIGS. 5D and 5N, and the second sensor response in water and animal B was shown in FIGS. 5O, 9A, 9B. All images were captured by a smartphone camera.

In some example implementations, to assess uplink performance, bit-error rate (BER) measurements are also performed at 50 mm depth and 360 samples per second (sps) f_{S} in deionized (DI) water and a muscle tissue-like phantom (see also FIGS. 7A-7C). The mote achieves a BER<10⁻⁵ (0 out of >10⁵ samples) and a modulation depth >10%, demonstrating a robust data uplink.

With respect to FIGS. 6A-6F, FIGS. 6A and 6B show an example system response measured at various dissolved oxygen (DO) concentrations; FIG. 6C shows an example Allan deviation of example data; FIG. 6D shows an example response to alternating streams of O₂ and N₂; FIG. 6E shows an example nonlinearity of a phase readout circuit; and FIG. 6F shows an example Stern-Volmer plot. To further explain, the mote shows a reversible and repeatable DO response, and its O₂ sensitivity is >0.5°/% (see, e.g., FIG. 6B); ϕ-resolution at f_{S}=360 sps is <0.38° in the O₂ range of 0-13.2%, giving an O₂ resolution <0.76% (see, e.g., FIG. 6C). Because the luminescence intensity (I) is a function of O₂ (I₀/I=1+K_{SV}[O₂]) and ϕ resolution is limited by jitter noise at the comparator output, the ϕ-resolution improves as DO concentration decreases. The total phase readout nonlinearity (NL), including the photodiode, TIA, comparator, and TDC, was evaluated using a function generator generating a modulated Δϕ to drive the μLED and computed using the endpoint method. The measured worst case NL is <0.27LSB (1)LSB=0.45° over the sensor operating ϕ range. The τ-based Stern-Volmer plot reveals nonlinear behavior, mainly due to inhomogeneous Ru-dye dispersion in the film.

FIGS. 7A-7C illustrate an example showing the advantages of the systems and methods disclosed herein over previously known systems. More specifically, in FIGS. 7A-7C:

[3] indicates measurements from L. Yao, et al., “Sensitivity-enhanced CMOS phase luminometry system using xerogel-based sensors,” IEEE TBioCAS, vol. 3, no. 5, pp. 304-311, October 2009;

[4] indicates measurements from W. P. Chan, et al., “A monolithically integrated pressure/oxygen/temperature sensing SoC for multimodality intracranial neuromonitoring,” IEEE JSSC, vol. 49, no. 11, pp. 2449-2461, November 2014; and

[5] indicates measurements from E. A. Johannessen, et al., “Implementation of multichannel sensors for remote biomedical measurements in a microsystems format,” IEEE Trans. Biomed. Eng., vol. 51, no. 3, pp. 525-535, March 2004.

### Example Effect of US Link Misalignment on the System Operation

The following section will describe an example effect of US link misalignment on the system operation. Although the use of acoustic waves, instead of near-field electromagnetic waves, enabled to power and communicate with the mm-scale wireless O₂ sensor at great depths ≥5 cm), it made the system sensitive to the US link alignment between the external transceiver and the wireless O₂ sensor. Therefore, it was helpful in understanding the impact of US link misalignment on the system operation.

The misalignment sensitivity of the system was evaluated by measuring the sensor V_{rect} and the uplink bit error rate (BER) (FIGS. 9C, 9D and 9E). The minimum V_{rect} necessary to turn on the μLED and hence to operate the sensor was ˜1.36 V (see FIG. 9G). The minimum acoustic intensity required to produce 1.36 V V_{rect} was 142 mW/cm², ˜19.7% of the FDA limit (derated ISPTA) of 720 mW/cm². This ˜5× acoustic intensity margin provided an ability to tolerate US link misalignment for proper sensor operation while keeping the intensity at safe levels.

Alignment measurements were performed in a water tank using a 25.4 mm-diameter transducer with a focal depth of 47.8 mm, generating acoustic pulses at 2 MHz with a fixed I_{SPTA} of 220 mW/cm² (FIG. 9C). When the sensor was operated on the center axis of the US beam with a zero angular offset, the system could robustly operate in a wide depth range of approximately 41-57 mm without sacrificing the BER performance (FIG. 9D); measured BERs at various depths within the operating depth range were below 10⁻⁵. When the sensor, positioned on the central axis of the acoustic field at the focal depth, was scanned transversely and angularly relative to the central axis of the sensor's piezo, the system also functioned properly with a 24° angular and 0.56 mm transverse offset relative to the beam central axis, at the expense of slight BER performance degradation at different angular and/or transverse offsets (FIGS 9E and 9F).

The link misalignment measurements showed that the system operation exhibited relatively high tolerance to the depth misalignment compared to the transverse misalignment, as the −3 dB depth-of-field (DOF: ˜12 mm) of the single-element focused transducer is substantially higher than its beam spot size (HPBW: ˜1.2 mm) at the focal depth (FIGS. 5T and 5U). In practice, a fine depth alignment could be achieved by using either an acoustic standoff pad and/or ultrasound gel or an external transducer with differing focal depths. Angular alignment within the operating angular misalignment range of ±24° could be performed by careful surgical placement of the sensor in tissue. Here, operation was relatively sensitive to transverse misalignment due to the transverse beam pattern produced by the external transducer. The transverse beam spot size at the desired focal length could be increased by optimizing the geometry of the acoustic lens that was built into the transducer. A custom-designed and built single-element transducer with a wider spot size could be used to reduce the system sensitivity to transverse misalignment, but at the expense of reduced US link power transfer efficiency.

Furthermore, when implemented, any of the methods and techniques described herein or portions thereof may be performed by executing software stored in one or more non-transitory, tangible, computer readable storage media or memories such as magnetic disks, laser disks, optical discs, semiconductor memories, biological memories, other memory devices, or other storage media, in a RAM or ROM of a computer or processor, etc.

Of course, the applications and benefits of the systems, methods and techniques described herein are not limited to only the above examples. Many other applications and benefits are possible by using the systems, methods and techniques described herein.

### EXEMPLARY EMBODIMENTS

Embodiment 1. A mote for measuring an O₂ level of a patient, the mote comprising:
a mote piezo configured to both send and receive ultrasound (US) waves;
a capacitor configured to be powered by the conversion of US waves received by the mote piezo to electrical energy; and
a luminescence sensor configured to be powered by the capacitor, wherein at least part of the luminescence sensor is optically isolated by an opaque material.

Embodiment 2. The mote of embodiment 1, wherein the opaque material is black silicon.

Embodiment 3. The mote of any one of embodiments 1-2, wherein the optical isolation is optical isolation between the at least part of the luminescence sensor and tissue of a patient.

Embodiment 4. The mote of any one of embodiments 1-3, wherein the luminescence sensor is entirely optically isolated from tissue of a patient.

Embodiment 5. The mote of any one of embodiments 1-4, wherein the luminescence sensor further comprises:
a light emitting diode (LED) configured for optical excitation;
a biocompatible film configured for encapsulation of O₂-sensitive luminescent ruthenium (Ru) dyes;
an optical filter; and
an integrated circuit (IC) with an integrated photodiode.

Embodiment 6. The mote of any one of embodiments 1-5, wherein:
the capacitor is part of a mote integrated circuit (IC);
the mote IC comprises: (i) an analog front-end including a transimpedance amplifier and comparator, (ii) a time-to-digital converter (TDC), (iii) a finite-state machine (FSM), (iv) a low dropout (LDO), (v) a voltage doubler, and (vi) a light emitting diode (LED) driver; and
the mote IC is configured to:
   in first phase: (i) power the capacitor by the conversion of the US waves received by the mote piezo to electrical energy, and (ii) duty cycle off at least one of the analog front-end, TDC, LDO, voltage doubler and LED driver; and
   in a second phase: receive an US data transmission.

Embodiment 7. The mote of any one of embodiments 1-6, wherein the luminescence sensor is configured to measure an O₂ level of a patient based on the US waves received by the mote piezo.

Embodiment 8. The mote of any one of embodiments 1-7, wherein the capacitor has a value of less than 100 nF.

Embodiment 9. The mote of any one of embodiments 1-8, wherein the capacitor has a value of 2.5 nF.

Embodiment 10. A method for measuring an O₂ level of a patient, the method comprising:
in a power up phase, powering a capacitor by receiving an ultrasound (US) signal; and
in a data transmission phase, receiving an US data transmission;
wherein, during the data transmission phase, at least one component of a mote is duty cycled off.

Embodiment 11. The method of embodiment 10, wherein the at least one component of the mote includes at least one of:
an analog front-end including a transimpedance amplifier and comparator;
a time-to-digital converter (TDC);
a low dropout (LDO);
a voltage doubler; and
a light emitting diode (LED) driver.

Embodiment 12. The method of any one of embodiments 10-11, wherein the at least one component of the mote includes all of:
an analog front-end including a transimpedance amplifier and comparator;
a time-to-digital converter (TDC);
a low dropout (LDO);
a voltage doubler; and
a light emitting diode (LED) driver.

Embodiment 13. The method of any one of embodiments 10-12, further comprising:
transmitting an electrical current generated from the received US data transmission to a luminescence sensor configured to measure the O₂ level of the patient;
modulating the electrical current based on the measured O₂ level;
transducing the modulated electrical current into an ultrasonic backscatter that encodes the measured O₂ level; and
emitting the ultrasonic backscatter to an interrogator.

Embodiment 14. The method of any one of embodiments 10-13, further comprising:
during the data transmission phase:
   transmitting an electrical current generated from the received US data transmission to a luminescence sensor configured to measure the O₂ level of the patient; and
   modulating the electrical current based on the measured O₂ level; and
during a backscatter phase:
   transducing the modulated electrical current into an ultrasonic backscatter that encodes the measured O₂ level; and
   emitting the ultrasonic backscatter to an interrogator.

Embodiment 15. The method of any one of embodiments 10-14, wherein during a backscatter phase:
the at least one component of the mote is duty cycled on; and
the capacitor discharges to power the at least one component of the mote.

Embodiment 16. The method of any one of embodiments 10-15, wherein:
the mote comprises a luminescence sensor configured to be powered by the capacitor; and
at least part of the luminescence sensor is optically isolated by an opaque material.

Embodiment 17. The method of any one of embodiments 10-16, wherein:
the mote comprises a luminescence sensor configured to be powered by the capacitor; and
at least part of the luminescence sensor is optically isolated by black silicon.

Embodiment 18. The method of any one of embodiments 10-17, wherein:
the mote comprises a luminescence sensor configured to be powered by the capacitor;
the entire luminescence sensor is optically isolated; and
at least part of the optical isolation is provided by black silicon.

Embodiment 19. The method of any one of embodiments 10-18, further comprising exciting an O₂-sensitive luminescent ruthenium (Ru) dye based on the received US data transmission.

Embodiment 20. A device for sending and receiving ultrasound (US) signals to a mote, the device comprising:
a piezo configured to send and receive ultrasound (US) waves;
an US interrogator configured to control the piezo to send and receive the US waves such that:
   in a power up phase: a power US transmission is made to the mote; and
   in a data transmission phase: a data US transmission is made to the mote.

Embodiment 21. The device of embodiment 20, wherein the US interrogator is configured to control the piezo to send and receive the US waves such that no data US transmission is made during the power up phase.

Embodiment 22. The device of any one of embodiments 20-21, wherein:
the US interrogator is further configured to receive US backscatter; and
the US interrogator is configured to analyze the US backscatter to determine a measured amount of O₂.

Embodiment 23. The device of any one of embodiments 20-22, wherein the US interrogator is further configured to charge a capacitor of the mote to a predetermined level by controlling the power US transmission.

Embodiment 24. The device of any one of embodiments 20-23, wherein the US interrogator is further configured to bring a voltage level of a low drop out (LDO) of the mote to a predetermined voltage level by controlling the power US transmission.

Embodiment 25. The device of any one of embodiments 20-24, wherein the US interrogator is further configured to, by controlling the power US transmission, bring:
a voltage level of an analog low drop out (A-LDO) of the mote to a predetermined analog VDD (A-VDD) voltage level; and
a voltage level of a digital low drop out (D-LDO) of the mote to a predetermined digital VDD (D-VDD) voltage level.

Embodiment 26. The device of any one of embodiments 20-25, wherein a luminescence sensor of the mote is optically isolated from a tissue of a patient.

Embodiment 27. The device of any one of embodiments 20-26, wherein the data US transmission is configured to cause a luminescence sensor of the mote to excite an O₂-sensitive luminescent ruthenium (Ru) dye.

Embodiment 28. A method for measuring an O₂ level of a patient using pulse-echo ultrasound (US) communication, the method comprising:
dividing data into a first data packet and a second data packet, wherein the first data packet includes most significant bits and the second data packet includes least significant bits;
in a first data transmission phase, transmitting the first data packet;
in a second data transmission phase, transmitting the second data packet; and
measuring the O₂ level of the patient according to the transmitted first and second data packets.

Embodiment 29. The method of embodiment 28, further comprising:
during a first receive backscatter phase, receiving backscatter of the first data packet; and
during a second receive backscatter phase, receiving backscatter from the second data packet.

Embodiment 30. The method of any one of embodiments 28-29, further comprising, prior to the first data transmission phase:
in a power up phase, powering a capacitor by transmitting an US signal.

Embodiment 31. The method of any one of embodiments 28-30, wherein a preamble precedes the most significant bits of the first data packet.

Embodiment 32. The method of any one of embodiments 28-31, wherein a postamble follows the least significant bits of the second data packet.

Embodiment 33. The method of any one of embodiments 28-32, wherein the first data packet and the second data packet are each 15 μs long.

Embodiment 34. The method of any one of embodiments 28-33, wherein:
the most significant bits of the first data packet are five bits; and
a one bit preamble precedes the most significant bits of the first data packet.

Embodiment 35. The method of any one of embodiments 28-34, wherein:
the least significant bits of the second data packet are five bits; and
a one bit postamble follows the least significant bits of the second data packet.

### INCORPORATION BY REFERENCE

Each of the following documents is incorporated by reference in their entirety:
U.S. Patent Application Publication No. 2019/0150883 entitled “IMPLANTS USING ULTRASONIC BACKSCATTER FOR DETECTING ELECTROPHYSIOLOGICAL SIGNALS;”
U.S. Patent Application Publication No. 2019/0150882 entitled “IMPLANTS USING ULTRASONIC BACKSCATTER FOR SENSING ELECTRICAL IMPEDANCE OF TISSUE;”
U.S. Patent Application Publication No. 2019/0150884 entitled “IMPLANTS USING ULTRASONIC WAVES FOR STIMULATING TISSUE;”
U.S Pat. No. 10,300,310 entitled “IMPLANTS USING ULTRASONIC BACKSCATTER FOR SENSING PHYSIOLOGICAL CONDITIONS;”
U.S Pat. No. 10,300,309 entitled “IMPLANTS USING ULTRASONIC BACKSCATTER FOR SENSING PHYSIOLOGICAL CONDITIONS;”
U.S. Patent Application Publication No. 2019/0150881 entitled “IMPLANTS USING ULTRASONIC BACKSCATTER FOR RADIATION DETECTION AND ONCOLOGY;” and
U.S Pat. No. 10,118,054 entitled “IMPLANTS USING ULTRASONIC BACKSCATTER FOR SENSING PHYSIOLOGICAL CONDITIONS.”

1. A mote for measuring an O₂ level of a patient, the mote comprising:
a mote piezo configured to both send and receive ultrasound (US) waves;
a capacitor configured to be powered by the conversion of US waves received by the mote piezo to electrical energy; and
a luminescence sensor configured to be powered by the capacitor, wherein at least part of the luminescence sensor is optically isolated by an opaque material.

2. The mote of claim 1, wherein the opaque material is black silicon.

3. The mote of claim 1, wherein the optical isolation is optical isolation between the at least part of the luminescence sensor and tissue of a patient.

4. The mote of claim 1, wherein the luminescence sensor is entirely optically isolated from tissue of a patient.

5. The mote of claim 1, wherein the luminescence sensor further comprises:
a light emitting diode (LED) configured for optical excitation;
a biocompatible film configured for encapsulation of O₂-sensitive luminescent ruthenium (Ru) dyes;
an optical filter; and
an integrated circuit (IC) with an integrated photodiode.

6. The mote of claim 1, wherein:
the capacitor is part of a mote integrated circuit (IC);
the mote IC comprises: (i) an analog front-end including a transimpedance amplifier and comparator, (ii) a time-to-digital converter (TDC), (iii) a finite-state machine (FSM), (iv) a low dropout (LDO), (v) a voltage doubler, and (vi) a light emitting diode (LED) driver; and
the mote IC is configured to:
in first phase: (i) power the capacitor by the conversion of the US waves received by the mote piezo to electrical energy, and (ii) duty cycle off at least one of the analog front-end, TDC, LDO, voltage doubler and LED driver; and
in a second phase: receive a US data transmission.

7. The mote of claim 1, wherein the luminescence sensor is configured to measure an O₂ level of a patient based on the US waves received by the mote piezo.

8. The mote of claim 1, wherein the capacitor has a value of less than 100 nF.

9. The mote of claim 1, wherein the capacitor has a value of 2.5 nF.

10. A method for measuring an O₂ level of a patient, the method comprising:
in a power up phase, powering a capacitor by receiving an ultrasound (US) signal; and
in a data transmission phase, receiving a US data transmission;
wherein, during the data transmission phase, at least one component of a mote is duty cycled off.

11. The method of claim 10, wherein the at least one component of the mote includes at least one of:
an analog front-end including a transimpedance amplifier and comparator;
a time-to-digital converter (TDC);
a low dropout (LDO);
a voltage doubler; and
a light emitting diode (LED) driver.

12. The method of claim 10, wherein the at least one component of the mote includes all of:
an analog front-end including a transimpedance amplifier and comparator;
a time-to-digital converter (TDC);
a low dropout (LDO);
a voltage doubler; and
a light emitting diode (LED) driver.

13. The method of claim 10, further comprising:
transmitting an electrical current generated from the received US data transmission to a luminescence sensor configured to measure the O₂ level of the patient;
modulating the electrical current based on the measured O₂ level;
transducing the modulated electrical current into an ultrasonic backscatter that encodes the measured O₂ level; and
emitting the ultrasonic backscatter to an interrogator.

14. The method of claim 10, further comprising:
during the data transmission phase:
transmitting an electrical current generated from the received US data transmission to a luminescence sensor configured to measure the O₂ level of the patient; and
modulating the electrical current based on the measured O₂ level; and
during a backscatter phase:
transducing the modulated electrical current into an ultrasonic backscatter that encodes the measured O₂ level; and
emitting the ultrasonic backscatter to an interrogator.

15. The method of claim 10, wherein during a backscatter phase:
the at least one component of the mote is duty cycled on; and
the capacitor discharges to power the at least one component of the mote.

16. The method of claim 10, wherein:
the mote comprises a luminescence sensor configured to be powered by the capacitor; and
at least part of the luminescence sensor is optically isolated by an opaque material.

17. The method of claim 10, wherein:
the mote comprises a luminescence sensor configured to be powered by the capacitor; and
at least part of the luminescence sensor is optically isolated by black silicon.

18. The method of claim 10, wherein:
the mote comprises a luminescence sensor configured to be powered by the capacitor;
the entire luminescence sensor is optically isolated; and
at least part of the optical isolation is provided by black silicon.

19. The method of claim 10, further comprising exciting an O₂-sensitive luminescent ruthenium (Ru) dye based on the received US data transmission.

20. A device for sending and receiving ultrasound (US) signals to a mote, the device comprising:
a piezo configured to send and receive ultrasound (US) waves;
an US interrogator configured to control the piezo to send and receive the US waves such that:
in a power up phase: a power US transmission is made to the mote; and
in a data transmission phase: a data US transmission is made to the mote.

21. The device of claim 20, wherein the US interrogator is configured to control the piezo to send and receive the US waves such that no data US transmission is made during the power up phase.

22. The device of claim 20, wherein:
the US interrogator is further configured to receive US backscatter; and
the US interrogator is configured to analyze the US backscatter to determine a measured amount of O₂.

23. The device of claim 20, wherein the US interrogator is further configured to charge a capacitor of the mote to a predetermined level by controlling the power US transmission.

24. The device of claim 20, wherein the US interrogator is further configured to bring a voltage level of a low drop out (LDO) of the mote to a predetermined voltage level by controlling the power US transmission.

25. The device of claim 20, wherein the US interrogator is further configured to, by controlling the power US transmission, bring:
a voltage level of an analog low drop out (A-LDO) of the mote to a predetermined analog VDD (A-VDD) voltage level; and
a voltage level of a digital low drop out (D-LDO) of the mote to a predetermined digital VDD (D-VDD) voltage level.

26. The device of claim 20, wherein a luminescence sensor of the mote is optically isolated from a tissue of a patient.

27. The device of claim 20, wherein the data US transmission is configured to cause a luminescence sensor of the mote to excite an O₂-sensitive luminescent ruthenium (Ru) dye.

28. A method for measuring an O₂ level of a patient using pulse-echo ultrasound (US) communication, the method comprising:
dividing data into a first data packet and a second data packet, wherein the first data packet includes most significant bits and the second data packet includes least significant bits;
in a first data transmission phase, transmitting the first data packet;
in a second data transmission phase, transmitting the second data packet; and
measuring the O₂ level of the patient according to the transmitted first and second data packets.

29. The method of claim 28, further comprising:
during a first receive backscatter phase, receiving backscatter of the first data packet; and
during a second receive backscatter phase, receiving backscatter from the second data packet.

30. The method of claim 28, further comprising, prior to the first data transmission phase:
in a power up phase, powering a capacitor by transmitting an US signal.

31. The method of claim 28, wherein a preamble precedes the most significant bits of the first data packet.

32. The method of claim 28, wherein a postamble follows the least significant bits of the second data packet.

33. The method of claim 28, wherein the first data packet and the second data packet are each 15 μs long.

34. The method of claim 28, wherein:
the most significant bits of the first data packet are five bits; and
a one bit preamble precedes the most significant bits of the first data packet.

35. The method of claim 28, wherein:
the least significant bits of the second data packet are five bits; and
a one bit postamble follows the least significant bits of the second data packet.
